(19) United States
(12) Patent Application Publication
(10) Pub. No.: US 2022/0233711 A1
(43) Pub. Date: Jul. 28, 2022

(54) BISPECIFIC T-CELL ENGAGER WITH CLEAVABLE CYTOKINES FOR TARGETED IMMUNOTHERAPY

(71) Applicant: SHENZHEN ENDURING BIOTECH, LTD., Shenzhen (CN)

(72) Inventors: Shumin Liu, Shenzhen (CN); Dechun Wu, Shenzhen (CN); Yu Wen, Shenzhen (CN); Yang Lei, Shenzhen (CN); Weidong Lyu, Shenzhen (CN)

(73) Assignee: SHENZHEN ENDURING BIOTECH, LTD., Shenzhen (CN)

(21) Appl. No.: 17/612,052

(22) PCT Filed: May 15, 2020
(86) PCT No.: PCT/CN2020/090488

§ 371 Date: Nov. 17, 2021

(30) Foreign Application Priority Data
May 17, 2019 (CN) PCT/CN2018/087379

(51) Int. Cl. A61K 47/68; A61P 35/00

(52) U.S. Cl. CPC A61K 47/6851; A61K 47/6849; A61P 35/00; A61K 47/6813; A61K 47/6889

(57) ABSTRACT

A long acting modified T-cell engager bispecific antibody with cytokine caps that provides reduced toxicity and boosted anti-tumor activity is disclosed. Method of making the modified and cytokine capped Bi-specific T-cell engager antibody is also disclosed.

### FIELD OF INVENTION

The present invention relates to a bispecific antibody with cleavable immunocytokine caps aiming to reduce toxicity and improve efficacy. In particular, the invention relates to a long acting modified Bi-specific T-cell engager (BiTE) antibody conjugated with releasable cytokines.

### BACKGROUND OF INVENTION

The advances in cancer biology and tumorigenesis in the past two decades have witnessed many new and more effective therapies that have revolutionized the treatment of malignant cancers. Data from cancer immunotherapy have established that supplementing and augmenting existing antitumor immune responses offer great opportunities to potentiate durable remission in cancer. Among various recently FDA approved agents, the Bi-specific T-cell engager (BiTE) Blinatumomab (Blincyto®) represents a new therapeutic perspective due to its engineered structure and the clinical efficacy for relapsed or refractory B lineage leukemia or lymphoma. Blinatumomab is a fusion protein of two single-chain antibodies linked by a five-amino-acid chain with dual affinity for CD19 and CD3. The simultaneous binding to both CD3-expressing T cells and CD19-expressing malignant B cells activates and engage cytotoxic T cells to blinatumomab bound malignant B cells, resulting in the lysis of target CD19+ B cancerous cells.

Another category of recent development is the immune check point drugs targeting PD-1, PD-L1, CTLA-4 etc. that have shown to be helpful in treating several types of cancer, including melanoma of the skin, non-small cell lung cancer, kidney cancer, bladder cancer, head and neck cancers, and Hodgkin lymphoma. They are also being studied for use against many other types of cancer. However, despite huge success of these drugs, many cancer patients still don't respond to such treatments, therefore cannot benefit from these technologies as a result of many immunosuppressive mechanisms existing in solid tumors, such as T cell exhaustion, limited tumor infiltration lymphocytes (TIL) in particular CD8+ T cells, tumor penetration hurdles because of the nature of tumor microenvironment, etc. The presence of enough active CD8+ T cells in solid tumor is very critical to have desired therapeutic outcome for patients.

To further advance the technology, fusion of BiTE technology with a check-point drug is expected to improve current check-point single agent therapies because of its dual mechanism advantage of blocking immune check point signaling and redirecting cytotoxic T cell to tumor cells to augment anti-tumor immunity. In fact, the pre-clinical study using anti-CD3/anti-PDL1 BiTE has already showed superior antitumor activity comparing to the single agent of anti-PDL1. Unfortunately, since human healthy tissue and activated T cells may also express PDL1, the anti-CD3/anti-PDL1 BiTE may kill some of those cells, resulting in severe side effects and exhaustion of T cell reservoir. Therefore, there is an urgent need for a novel and better technology as disclosed in this invention.

### SUMMARY OF THE INVENTION

This invention addresses the aforementioned unmet needs by providing a long acting modified BiTE antibody conjugated with conditionally releasable cytokine molecules and related methods.

In one aspect, the invention provides a multi-specific molecule, conjugate, or compound of the Formula Ia

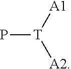

P can be a non-immunogenic polymer. T can be a trifunctional small molecule derived linker moiety and may have two or more functional groups that are capable of site-specific conjugation with two different proteins. A1 and A2 can be any two different or same proteins.

In particular, an aspect of the invention provides a conjugate of Formula Ib:

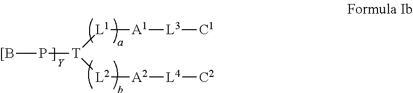

In the conjugate,

P can be a non-immunogenic polymer;

B can be H, a terminal capping group or void, said capping group selected from C₁₋₅₀ alkyl and aryl, wherein one or more carbons of said alkyl may be replaced with a heteroatom;

T can be a multi-functional linker having two, or more functional groups, wherein the linkage between T and (L¹)ₐ and the linkage between T and (L²)_{b} could be the same or different;

each of L¹ and L² can be independently a bifunctional linker, or a peptide liner;

L³ and L⁴ can be independently enzyme cleavable substrate, L³ and L⁴ could be the same or different; L³ or L⁴ could also be null;

a and b can each be an integer selected from 0-10, inclusive;

A¹ and A² can be any two different or same proteins. For example, A¹ and A² can be different from each other and each of A¹ and A² independently may comprise an antibody fragment, single chain antibody, or any other antigen binding portion or combination thereof; or A¹ and A² can be the same and both can be multispecific antigen binding protein; and

C¹ and C² can be any two different or same cytokine proteins. For example, C¹ and C² can be different from each other and each of C¹ and C² independently may comprise cytokines or null, and

y can be an integer selected from 1-10.

At least one of the proteins may comprise a recognition binding moiety. For example, A¹ may comprise a first recognition binding moiety and A² may comprise a second recognition binding moiety. The two different proteins can be two different antibodies or antigen-binding portions thereof. In one example, the two antibodies are respectively an anti-CD3 antibody that binds to a protein on cytotoxic T cell and an anti-PD-L1 antibody that binds to an antigen on cancer cell. The two antibodies can be single chain antibodies (SCA or scFv).

The non-immunogenic polymer can be selected from the group consisting of polyethylene glycol (PEG), dextrans, carbohydrate-base polymers, polyalkylene oxide, polyvinyl alcohols, hydroxypropyl-methacrylamide (HPMA), and a co-polymer thereof. Preferably, the non-immunogenic polymer is PEG, such as a branched PEG or a linear PEG or a multi-arm PEG. In that case, at least one terminal of linear PEG or branch PEG can be capped with H, methyl or low molecule weight alkyl group. The total molecule weight of the PEG can be 3,000 to 100,000 Daltons, e.g., 5,000 to 80,000, 10,000 to 60,000, and 20,000 to 40,000 Daltons. The PEG can be linked to a multifunctional moiety either through a permanent bond or a cleavable bond.

The functional groups (e.g., two site-specific conjugation functional groups) that form linkages within (L¹)ₐ or (L²)_{b} or between (L¹)ₐ and protein A¹ or between (L²)_{b} and protein A² can be selected from the group consisting of thiol, maleimide, 2-pyridyldithio variant, aromatic sulfone or vinyl sulfone, acrylate, bromo or iodo acetamide, azide, alkyne, dibenzocyclooctyl (DBCO), carbonyl, 2-amino-benzaldehyde or 2-amino-acetophenone group, hydrazide, oxime, potassium acyltrifluoroborate, O-carbamoylhydroxylamine, trans-cyclooctene, tetrazine, triarylphosphine, etc.

In some embodiments, one of (L¹)ₐ and (L²)_{b} can comprise a linkage formed from azide and alkyne; the other of the (L¹)ₐ and (L²)_{b} can comprise a linkage formed from maleimide and thiol. In some examples, the alkyne can be dibenzocyclooctyl (DBCO). In others, T can be lysine, P can be PEG, and y can be 1, while the alkyne can be dibenzocyclooctyl (DBCO). In some embodiments, one of A¹ and A² can be derived from an azide tagged antibody, antibody fragment, or single chain antibody, wherein the azide can be conjugated to an alkyne in the respective (L′)ₐ or (L²)_{b}; the other of A¹ and A² can be derived from a thiol tagged antibody, antibody fragment or single chain antibody, wherein the thiol can be conjugated to a maleimide in the respective (L¹)ₐ or (L²)_{b}.

In some embodiments, (L¹)ₐ, (L²)_{b} and T are independently a peptide linker that contains no more than 25 amino acids. In other embodiments, T is derived from Cysteine, Lysine, Asparagine, Aspartic, Glutamic acid, Glutamine, Histidine, Serine, Threonine, Tryptophan, Tyrosine or any unnatural amino acid such as genetically-encoded amino acid with alkene handle, para-acetyl-phenylalanine etc.

In some embodiments, L³ and L⁴ can be independently cleavable substrates of proteases in tumor extracellular matrix, more specifically matrix metalloproteinases (MMPs), urokinase plasminogen Activator (uPA) etc. L³ and L⁴ can be the same or different, or the combinations of several cleavable substrates to speed up release of T-cell engager at tumor site. Examples of the protease include Collagenases (e.g., MMP-1, MMP-8, MMP-13), Gelatinases (e.g., gelatinase A and gelatinase B), matrilysins (e.g., matrilysin-1 and matrilysin-2), MMP-12, membrane type MMPs (e.g., MMT-14, MMP-15, MMP-16, MMP-17, MMP-24, and MMP-25), Stromelysins (e.g., Stromelysin-1/MMP_3, Stromelysin-2/MMP-10, and Stromelysin-3/MMP-11), MMP-21, MMP-27, a disintegrin and metalloproteinase with thrombospondin motifs (ADAMTs) (e.g., ADAMTS-1, ADAMTS-2 ADAMTS-3, ADAMTS-4, ADAMTS-5, ADAMTS-6, ADAMTS-8, ADAMTS-9, ADAMTS-12, ADAMTS-13, ADAMTS-17, and ADAMTS-18), Procathepsin B, cathepsin B, cathepsin S, cathepsin B, caspases (e.g., caspase 1, caspase 2, caspase 3, caspase 4, caspase 5, caspase 6, caspase 7, caspase 8, caspase 9, caspase 1,0 caspase 11, caspase 12, and caspase 13), Chondroitinase, Hyaluronidase, uPA, and tPA.

In some embodiments, C¹ and C² independently may comprise cytokine such as IL2v, IL10, etc. C¹ and C² can be the same or different, or the combination of several cytokines with a L³ or L⁴ in between.

The above-described multi-specific molecule or compound can be made according to a method comprising: (i) preparing a non-immunogenic polymer with terminal bi-functional groups capable of site-specific conjugation with two different proteins or their modified forms; and (ii) stepwise site-specific conjugating the non-immunogenic polymer with two different proteins or their modified forms to form a compound of Formula Ia or Ib. In some examples, before the preparing step, the proteins can be modified with a small molecule linker first.

A related aspect provide a chimeric construct of Formula II,

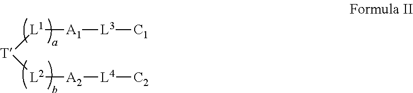

wherein:

A₁ and A₂ are two different antibodies, antibody fragments or single chain antibodies or other forms of antibodies or any combination thereof,

C₁ and C₂ are each a cleavable cap;

L³ and L⁴ are each an enzyme cleavable substrate or null;

L¹ and L² are each independently a bifunctional linker;

a and b can each be an integer selected from 0-10, inclusive;

and

T′ is a linker moiety.

The invention also provides a pharmaceutical formulation comprising the multi-specific molecule or compound described above and a pharmaceutically acceptable carrier. The invention further provides a method of treating a disease in a subject in need thereof comprising administering an effective amount of the multi-specific molecule or compound described above.

The details of one or more embodiments of the invention are set forth in the description below. Other features, objectives, and advantages of the invention will be apparent from the description and from the claims.

### DESCRIPTION OF THE DRAWINGS

FIG. 1 schematically illustrates a reaction scheme of preparing 30kmPEG-Lys(maleimide)-DBCO described in Example 1.

FIG. 2 schematically illustrates a reaction scheme of preparing 30kmPEG-(SCAPDL1IL10)SCACD3IL12 described in Example 3.

FIG. 3 shows the synthesis of PEGylated JY101A and JY101P.

FIG. 4 shows that 0.66 ug of JY101-AC fusion protein could be completely digested within 30 minutes by 33 ng of MMP14.

FIG. 5 shows that uPA digested JY101-AC is very potent in lysing PD-L1 expressing MDA-MB-231 cells in the presence of effector T cells. At the concentration of 1 ng/ml (E:T ratio of 2:1), the cytotoxicity of uPA digested JY101-AC reached as high as 75%.

FIG. 6 shows significantly higher cytotoxicity for JY101-AC (uPA digested) than JY101-PC at the low doses.

FIG. 7 shows cytotoxic synergy of JY101AC in vitro.

FIG. 8 shows a summary of cytotoxic synergy of JY101AC in vitro.

### DETAILED DESCRIPTION OF THE INVENTION

Cancer can be considered the consequential result of tumorous cells escaping from immunosurveillance. Manipulation of human immune system to re-engage cytoxic T cells to kill cancer has been greatly appreciated in the last two decades exemplified with the development of BiTE prototype compound Blinatomomab which has shown to be effective in treatment of cancer patients and proved by FDA as the first BiTE bispecific antibody.

Blinatumomab is a bispecific fusion antibody for treatment of cancer. It is made up of two single-chain monoclonal antibodies against CD19 and CD3 respectively. However, similar to other recombinant proteins, the Blinatumomab is cleared very quickly during blood circulation and must be administered by a continuous intravenous infusion for 4 weeks (24 hours a day, 7 days a week) with a portable mini-pump. This special drug administration has been a great challenge for patients to comply with, particularly for young children. Additionally, high chance of infection or even deadly infection has put these patients at great risk.

Many new BiTE bispecific antibody developments have been made in the last few years with the goal of increasing the circulation half-life and improving efficacy. For example, WO2018075308, which is incorporated herein by reference, disclosed a novel strategy to PEGylate two single chain antibodies to form a unique BiTE format.

PEGylation, as one of the most successful protein modification strategies, has been used extensively in pharmaceutical industry. It is well known that the conjugation of PEG with therapeutic molecules such as proteins and polypeptides could extend the circulation half-life and improve the pharmacokinetic and pharmacodynamic properties for these medicines. In this invention, PEGylated single-chain bispecific antibodies capped with conditionally releasable cytokines was made to address unmet medical needs.

### Powerful BiTE Technology

BiTE bispecific antibody can activate T cells directly through the CD3 complex which is downstream of TCR (T cell receptor) on the T cell activation pathway. Therefore the function of BiTEs is independent of T cell receptor specificity, MHC restriction, and costimulatory signals. Typical BiTEs are relatively small in molecular size (˜55 kD) which allows their two arms effectively bridging T cells to targeted cells to form an immunological synapse. The formation of an immunological synapse favors T cell activation and cytotoxic effect for tumor cells killing through a granzyme and perforin-mediated process [8], which is a common mechanism to all cytotoxic T cells activated by antigens conventionally. The approach of engaging cytotoxic T cell to build an immune synape to kill cancer has been proved to be very successful. Approximately 50% of clinical development of bispecific antibodies fall in this category currently.

### T Cell Reinvigoration

Cancer is the result of overwhelmed growth of abnormal tumor cells despite the presence of immune system. Tumor-infiltrating lymphocytes (TILS) at tumor site are usually dysfunctional and are predominantly considered “exhausted” because of the immunosuppression from the tumor cells or in the tumor micro-environment.

Numerous studies have confirmed that tumor cells are capable of developing strong immune suppression to patient immune system, which counteracts the cancer killing potency of activated T cells. Tumor-induced immune suppression is mediated by suppressive cell populations including myeloid-derived suppressor cells (MDSC) and T regulatory cells, as well as by checkpoints which cause T cell anergy and apoptosis. Reinvigorating T cells is critical to reboot immune system to fight against cancer.

### Checkpoints of Immune System

Research on interactions of immune surveillance and tumor development has advanced to the discovery of immune checkpoints such as PD-1, PD-L1, CTLA4 etc., which have been used in cancer therapy and shown clinical benefits. PD-L1, for example, forms an immunosuppressive axis with its receptor PD-1 on T cells to prevent over activation of the immune system, which is a powerful mechanism. Unfortunately this powerful mechanism could be hijacked by tumor cells to escape from immune surveillance. Humanized anti-PD-L1 monoclonal antibodies have been developed exactly to address such problems and already approved by FDA for immune checkpoint blockade therapy for the treatment of variety of cancers.

With the success of anti-PD-L1 monoclonal antibody therapies, it is feasible that the single chain anti-PD-L1 would also retain its therapeutic property. Besides, PD-L1 is prevalently expressed in a variety of cancer types, e.g., PD-L1 positive tumor specimens range from 38 to 100% in melanoma, and 21 to 95% in NSCLC respectively, therefore PD-L1 makes a very good tumor target for the single chain anti-CD3/anti-PDL1 bispecific antibody for the treatment of cancer patients currently not responding to PD-L1 monotherapy.

### Dual Mechanisms of CD3/PD-L1

Anti-PD-L1 single agent therapies could restore latent anti-tumor immunity and generate clinical response of 43% in melanoma, and approximately 20% in advanced NSCLC. Yet some patients do not respond to anti-PD-L1 single agent therapy even though the tumor specimens show PD-L1 positive. CD3/PDL1 bispecific antibodies are expected to improve anti-PD-L1 single agent therapies with dual mechanisms of blocking immune check point signaling and redirecting cytotoxic T cell to tumor cells to augment anti-tumor immunity, therefore, CD3/PDL1 bispecific antibodies are expected to overcome some primary resistance of anti-PD-L1 single agent therapy.

### Toxicity of Anti-CD3/Anti-PD-L1 BiTE

As tumors grow, PD-L1 expression is up regulated in stromal cells, antigen presenting cells, tumor filtrating myeloid cells and tumor vascular endothelial cells etc. The up-regulation of PD-L1 in those cells will confer immunosuppression to mediate immune tolerance and promote angiogenesis for tumor growth. The CD3/PD-L1 bispecific antibody would induce efficient removal of such immunosuppressive and pro-angiogenesis cells, which in turn would result in additive or even synergistic effect in boosting anti-tumor immunity and increasing therapeutic response. The study using anti-CD3/anti-PDL1 BiTE has already showed superior antitumor activity comparing to the single agent of anti-PDL1. However, healthy tissue and activated T cells may also express PD-L1, therefore the anti-CD3/anti-PDL1 BiTE may also kill some of those cells, resulting in server side effects and exhaustion of T cell reservoir and reduced the number of effector T cells.

### High Density of Active CD8+ T Cells Required

Despite of a variety of immunosuppression mechanisms during tumor growth, the presence of active lymphocytes or tumor-infiltrating lymphocytes (TILs) (mainly T cells) in tumor has been correlated with a good prognosis following treatment. A meta-analysis study revealed that high level of CD8+ and CD3+ T cells infiltration in tumor stroma or tumor nest showed better overall survival in lung cancer patients, whereas high density of FOXP3+ T cells, Treg infiltration in tumor stroma functioned as a negative prognostic factor. Clinical studies from other meta-analyses showed similar results for patients with esophagus cancer, hepatocellular carcinoma patients, breast cancer, gastric cancer and ovarian cancer, all of which confirmed that high infiltration of CD8+ TILs corresponded to better overall survival. It is also reported that CD3+ positive tumor infiltration lymphocytes were strongly associated with positive prognosis for patients with gastric cancer. Therefore, increased numbers of TILs particularly CD8+ T cells with T cell growth factors such as IL-2 or IL-10 are important to increase overall survival in cancer treatment.

### T Cell Growth Factors IL-2 and IL-10

IL-2 was identified in 1976 as a T cell growth factor and later approved for treatment of patients with metastatic melanoma and renal cell carcinoma with beneficial results in a subset of patients. IL-2 cytokine displays multiple immunological effects and acts by binding to the IL-2 receptor (IL-2R). The association of IL-2Rα (CD25), IL-2Rβ (CD122), and IL-2Rγ (CD132) subunits results in the trimeric high affinity IL-2Rαβγ. CD25 confers high affinity binding to IL-2, whereas the β and γ subunits (expressed on natural killer (NK) cells, monocytes, macrophages and resting CD4⁺ and CD8⁺ T cells) mediate signal transduction. It appears that the expression of CD25 is essential for the expansion of immunosuppressive regulatory T cells (Treg). On the other hand, cytolytic CD8⁺ T and NK cells can proliferate and kill target cells only by IL-2Rβγ engagement and in the absence of CD25. Therefore, the IL-2 cytokine acts as a master activating factor for helper/regulatory T cell and NK cell proliferation, differentiation and as a relevant mediator for pro- and anti-inflammatory immune responses. Administration of high-dose IL-2 can be associated with relevant adverse effects that include the vascular leakage syndrome, fever, chills, malaise, hypotension, organ dysfunction and cytopenia, which have been well-reviewed previously. Low-doses of IL-2 lead to the preferential expansion of Treg cells which is an undesired effect in anti-cancer immunotherapy. To overcome the toxicity related to the systemic administration of IL-2 at low dose, one example is to use a variant form of interleukin-2 (IL-2v), which cannot bind to IL-2 receptor-alpha (CD25, IL2Ra), therefore does not activate immunosuppressive regulatory T-cells (Tregs), but only exerts potential immunostimulating effect.

Recent discoveries show that IL-10 also activated and expanded tumor-resident CD8⁺ T cells. Pegilodecakin (a PEGylated IL-10) was evaluated across multiple advanced solid tumors in a large phase 1/1b trial alone and in combination with chemotherapy or anti-PD-1 antibodies. Study showed Pegilodecakin monotherapy had immunologic and clinical activity in renal cell carcinoma (RCC) and uveal melanoma. In combination with anti-PD-1 checkpoint inhibitors, study showed that pegilodecakin increased the responses in RCC and lung cancer with efficacy agnostic to PD-L1 status and tumor mutational burden.

### Antibodies

The invention disclosed herein involves antibodies. As used herein, “antibody” is used in the broadest sense and specifically covers monoclonal antibodies (including full length monoclonal antibodies), polyclonal antibodies, multispecific antibodies (e.g., bispecific antibodies), and antibody fragments so long as they exhibit the desired biological activity.

### Fragment

In certain embodiments, an antibody provided herein is an antibody fragment. Antibody fragments include, but are not limited to, Fab, Fab′, Fab′-SH, F(ab′)₂, Fv, and scFv fragments, and other fragments described below, e.g., diabodies, triabodies tetrabodies, and single-domain antibodies.

Diabodies are antibody fragments with two antigen-binding sites that may be bivalent or bispecific. Triabodies and tetrabodies are also described in literature.

Single-domain antibodies are antibody fragments comprising all or a portion of the heavy chain variable domain or all or a portion of the light chain variable domain of an antibody. In certain embodiments, a single-domain antibody is a human single-domain antibody in reports such as U.S. Pat. No. 6,248,516, the entire disclosure of which is incorporated herein by reference.

Antibody fragments can be made by various techniques, including but not limited to proteolytic digestion of an intact antibody as well as production by recombinant host cells (e.g., E. coli or phage), as described herein.

### Chimeric and Humanized Antibodies

In certain embodiments, an antibody provided herein is a chimeric antibody. In one example, a chimeric antibody comprises a non-human variable region (e.g., a variable region derived from a mouse, rat, hamster, rabbit, or non-human primate, such as a monkey) and a human constant region. In a further example, a chimeric antibody is a “class switched” antibody in which the class or subclass has been changed from that of the parent antibody. Chimeric antibodies include antigen-binding fragments thereof.

In certain embodiments, a chimeric antibody is a humanized antibody. Typically, a non-human antibody is humanized to reduce immunogenicity to humans, while retaining the specificity and affinity of the parental non-human antibody. Generally, a humanized antibody comprises one or more variable domains in which HVRs, e.g., CDRs, (or portions thereof) are derived from a non-human antibody, and FRs (or portions thereof) are derived from human antibody sequences. A humanized antibody optionally will also comprise at least a portion of a human constant region. In some embodiments, some FR residues in a humanized antibody are substituted with corresponding residues from a non-human antibody (e.g., the antibody from which the HVR residues are derived), e.g., to restore or improve antibody specificity or affinity.

Humanized antibodies and methods of making them have been described in references such as U.S. Pat. Nos. 5,821,337, 7,527,791, 6,982,321, and 7,087,409, the entire disclosure of all of these patents are herein incorporated by reference.

Human framework regions that may be used for humanization include but are not limited to: framework regions selected using the “best-fit” method; framework regions derived from the consensus sequence of human antibodies of a particular subgroup of light or heavy chain variable regions; human mature (somatically mutated) framework regions or human germline framework regions; and framework regions derived from screening FR libraries.

### Human Antibodies

In certain embodiments, an antibody provided herein is a human antibody. Human antibodies can be produced using various techniques known in the art or using techniques described herein.

Human antibodies may be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic mice, the endogenous immunoglobulin loci have generally been inactivated. For review of methods for obtaining human antibodies from transgenic animals, see e.g., U.S. Pat. Nos. 6,075,181 and 6,150,584 describing XENOMOUSE technology; U.S. Pat. No. 5,770,429 describing HUMAB technology; U.S. Pat. No. 7,041,870 describing K-M MOUSE technology, and U.S. Patent Application Publication No. US 2007/0061900, describing VELOCIMOUSE technology); the entire disclosure of these patents and patent application are incorporated by reference. Human variable regions from intact antibodies generated by such animals may be further modified, e.g., by combining with a different human constant region.

Human antibodies can also be made by hybridoma-based methods. Human myeloma and mouse-human heteromyeloma cell lines for the production of human monoclonal antibodies have been described. An exemplary procedure is provided in U.S. Pat. No. 7,189,826 (describing production of monoclonal human IgM antibodies from hybridoma cell lines), the entire disclosure of which is incorporated by reference. Human hybridoma technology (Trioma technology) is also well known in the art.

Human antibodies may also be generated by isolating Fv clone variable domain sequences selected from human-derived phage display libraries. Such variable domain sequences may then be combined with a desired human constant domain. Techniques for selecting human antibodies from antibody libraries are described below.

Antibodies of the invention may be isolated by screening combinatorial libraries for antibodies with the desired activity or activities. For example, a variety of methods are known in the art for generating phage display libraries and screening such libraries for antibodies possessing the desired binding characteristics. Such methods are well known if the art.

In certain phage display methods, repertoires of VH and VL genes are separately cloned by polymerase chain reaction (PCR) and recombined randomly in phage libraries, which can then be screened for antigen-binding phage as known in the art. Phage typically display antibody fragments, either as single-chain Fv (scFv) fragments or as Fab fragments. Libraries from immunized sources provide high-affinity antibodies to the immunogen without the requirement of constructing hybridomas. Alternatively, the naive repertoire can be cloned (e.g., from human) to provide a single source of antibodies to a wide range of non-self and also self-antigens without any immunization as well known in the art. Finally, naive libraries can also be made synthetically by cloning unrearranged V-gene segments from stem cells, and using PCR primers containing random sequence to encode the highly variable CDR3 regions and to accomplish rearrangement in vitro, as well known in the art. Patent publications describing human antibody phage libraries include, for example: U.S. Pat. No. 5,750,373, and US Patent Publication Nos. 2005/0079574, 2005/0119455, 2005/0266000, 2007/0117126, 2007/0160598, 2007/0237764, 2007/0292936, and 2009/0002360, the entire disclosure of these patents and patent applications are incorporated by reference. Antibodies or antibody fragments isolated from human antibody libraries are considered human antibodies or human antibody fragments herein.

### Variants

In certain embodiments, amino acid sequence variants of the antibodies provided herein are contemplated. For example, it may be desirable to improve the binding affinity and/or other biological properties of the antibody. Amino acid sequence variants of an antibody may be prepared by introducing appropriate modifications into the nucleotide sequence encoding the antibody, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, e.g., antigen-binding.

### Substitution, Insertion, and Deletion Variants

In certain embodiments, antibody variants having one or more amino acid substitutions are provided. Sites of interest for substitutional mutagenesis include the HVRs and FRs. Conservative substitutions are defined herein. Amino acid substitutions may be introduced into an antibody of interest and the products screened for a desired activity, e.g., retained/improved antigen binding, decreased immunogenicity, or improved ADCC or CDC.

Accordingly, an antibody of the invention can comprise one or more conservative modifications of the CDRs, heavy chain variable region, or light variable regions described herein. A conservative modification or functional equivalent of a peptide, polypeptide, or protein disclosed in this invention refers to a polypeptide derivative of the peptide, polypeptide, or protein, e.g., a protein having one or more point mutations, insertions, deletions, truncations, a fusion protein, or a combination thereof. It retains substantially the activity to of the parent peptide, polypeptide, or protein (such as those disclosed in this invention). In general, a conservative modification or functional equivalent is at least 60% (e.g., any number between 60% and 100%, inclusive, e.g., 60%, 70%, 75%, 80%, 85%, 90%, 95%, 96%, 97%, 98%, and 99%) identical to a parent sequence such as one of SEQ ID NOs: 1-5. Accordingly, within scope of this invention are heavy chain variable region or light variable regions having one or more point mutations, insertions, deletions, truncations, a fusion protein, or a combination thereof, as well as antibodies having the variant regions.

As used herein, the percent homology between two amino acid sequences is equivalent to the percent identity between the two sequences. The percent identity between the two sequences is a function of the number of identical positions shared by the sequences (i.e., % homology=#of identical positions/total #of positions×100), taking into account the number of gaps, and the length of each gap, which need to be introduced for optimal alignment of the two sequences. The comparison of sequences and determination of percent identity between two sequences can be accomplished using a mathematical algorithm, as described in the non-limiting examples below.

The percent identity between two amino acid sequences can be determined using the algorithm of E. Meyers and W. Miller (Comput. Appl. Biosci., 4:11-17 (1988)) which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch (J. Mol. Biol. 48:444-453 (1970)) algorithm which has been incorporated into the GAP program in the GCG software package (available at www.gcg.com), using either a Blossum 62 matrix or a PAM250 matrix, and a gap weight of 16, 14, 12, 10, 8, 6, or 4 and a length weight of 1, 2, 3, 4, 5, or 6.

Additionally or alternatively, the protein sequences of the present invention can further be used as a “query sequence” to perform a search against public databases to, for example, identify related sequences. Such searches can be performed using the XBLAST program (version 2.0) of Altschul, et al. (1990) J. Mol. Biol. 215:403-10. BLAST protein searches can be performed with the XBLAST program, score=50, wordlength=3 to obtain amino acid sequences homologous to the antibody molecules of the invention. To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g., XBLAST and NBLAST) can be used. (See www.ncbi.nlm.nih.gov).

As used herein, the term “conservative modifications” refers to amino acid modifications that do not significantly affect or alter the binding characteristics of the antibody containing the amino acid sequence. Such conservative modifications include amino acid substitutions, additions and deletions. Modifications can be introduced into an antibody of the invention by standard techniques known in the art, such as site-directed mutagenesis and PCR-mediated mutagenesis. Conservative amino acid substitutions are ones in which the amino acid residue is replaced with an amino acid residue having a similar side chain. Families of amino acid residues having similar side chains have been defined in the art. These families include:

amino acids with basic side chains (e.g., lysine, arginine, histidine),

acidic side chains (e.g., aspartic acid, glutamic acid),

uncharged polar side chains (e.g., glycine, asparagine, glutamine, serine, threonine, tyrosine, cysteine, tryptophan),

nonpolar side chains (e.g., alanine, valine, leucine, isoleucine, proline, phenylalanine, methionine),

beta-branched side chains (e.g., threonine, valine, isoleucine) and

aromatic side chains (e.g., tyrosine, phenylalanine, tryptophan, histidine).

Non-conservative substitutions will entail exchanging a member of one of these classes for another class.

An exemplary substitutional variant is an affinity matured antibody, which may be conveniently generated, e.g., using phage display-based affinity maturation techniques well known in the art. Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to an enzyme (e.g., for ADEPT) or a polypeptide which increases the serum half-life of the antibody.

### Fc Region Variants

The variable regions of the antibody described herein can be linked (e.g., covalently linked or fused) to an Fc, e.g., an IgG1, IgG2, IgG3 or IgG4 Fc, which may be of any allotype or isoallotype, e.g., for IgG1: G1m, G1m1(a), G1m2(x), G1m3(f), G1m17(z); for IgG2: G2m, G2m23(n); for IgG3: G3m, G3m21(g1), G3m28(g5), G3 m1 1(b0), G3m5(b1), G3m13(b3), G3m14(b4), G3m10(b5), G3m15(s), G3m16(t), G3m6(c3), G3m24(c5), G3m26(u), G3m27(v); and for K: Km, Km1, Km2, Km3 (see, e.g., Jefferies et al. (2009) mAbs 1: 1). In certain embodiments, the antibodies variable regions described herein are linked to an Fc that binds to one or more activating Fc receptors (FcγI, FcγIIa or FcγIIIa), and thereby stimulate ADCC and may cause T cell depletion. In certain embodiments, the antibody variable regions described herein are linked to an Fc that causes depletion.

In certain embodiments, the antibody variable regions described herein may be linked to an Fc comprising one or more modification, typically to alter one or more functional properties of the antibody, such as serum half-life, complement fixation, Fc receptor binding, and/or antigen-dependent cellular cytotoxicity. Furthermore, an antibody described herein may be chemically modified (e.g., one or more chemical moieties can be attached to the antibody) or be modified to alter its glycosylation, to alter one or more functional properties of the antibody. The numbering of residues in the Fc region is that of the EU index of Kabat.

The Fc region encompasses domains derived from the constant region of an immunoglobulin, preferably a human immunoglobulin, including a fragment, analog, variant, mutant or derivative of the constant region. Suitable immunoglobulins include IgG1, IgG2, IgG3, IgG4, and other classes such as IgA, IgD, IgE and IgM, The constant region of an immunoglobulin is defined as a naturally-occurring or synthetically-produced polypeptide homologous to the immunoglobulin C-terminal region, and can include a CH1 domain, a hinge, a CH2 domain, a CH3 domain, or a CH4 domain, separately or in combination. In some embodiments, an antibody of this invention has an Fc region other than that of a wild type IgA1. The antibody can have an Fc region from that of IgG (e.g., IgG1, IgG2, IgG3, and IgG4) or other classes such as IgA2, IgD, IgE and IgM. The Fc can be a mutant form of IgA1.

The constant region of an immunoglobulin is responsible for many important antibody functions including Fc receptor (FcR) binding and complement fixation. There are five major classes of heavy chain constant region, classified as IgA, IgG, IgD, IgE, IgM, each with characteristic effector functions designated by isotype. For example, IgG is separated into four subclasses known as IgG1, IgG2, IgG3, and IgG4.

Ig molecules interact with multiple classes of cellular receptors. For example IgG molecules interact with three classes of Fcγ receptors (FcγR) specific for the IgG class of antibody, namely FcγRI, FcγRII, and FcγRIII. The important sequences for the binding of IgG to the FcγR receptors have been reported to be located in the CH2 and CH3 domains. The serum half-life of an antibody is influenced by the ability of that antibody to bind to an Fc receptor (FcR).

In certain embodiments, the Fc region is a variant Fc region, e.g., an Fc sequence that has been modified (e.g., by amino acid substitution, deletion and/or insertion) relative to a parent Fc sequence (e.g., an unmodified Fc polypeptide that is subsequently modified to generate a variant), to provide desirable structural features and/or biological activity. For example, one may make modifications in the Fc region in order to generate an Fc variant that (a) has increased or decreased antibody-dependent cell-mediated cytotoxicity (ADCC), (b) increased or decreased complement mediated cytotoxicity (CDC), (c) has increased or decreased affinity for C1q and/or (d) has increased or decreased affinity for a Fc receptor relative to the parent Fc. Such Fc region variants will generally comprise at least one amino acid modification in the Fc region. Combining amino acid modifications is thought to be particularly desirable. For example, the variant Fc region may include two, three, four, five, etc. substitutions therein, e.g., of the specific Fc region positions identified herein.

A variant Fc region may also comprise a sequence alteration wherein amino acids involved in disulfide bond formation are removed or replaced with other amino acids. Such removal may avoid reaction with other cysteine-containing proteins present in the host cell used to produce the antibodies described herein. Even when cysteine residues are removed, single chain Fc domains can still form a dimeric Fc domain that is held together non-covalently. In other embodiments, the Fc region may be modified to make it more compatible with a selected host cell. For example, one may remove the PA sequence near the N-terminus of a typical native Fc region, which may be recognized by a digestive enzyme in E. coli such as proline aminopeptidase. In other embodiments, one or more glycosylation sites within the Fc domain may be removed. Residues that are typically glycosylated (e.g., asparagine) may confer cytolytic response. Such residues may be deleted or substituted with unglycosylated residues (e.g., alanine) In other embodiments, sites involved in interaction with complement, such as the C1q binding site, may be removed from the Fc region. For example, one may delete or substitute the EKK sequence of human IgG1. In certain embodiments, sites that affect binding to Fc receptors may be removed, preferably sites other than salvage receptor binding sites. In other embodiments, an Fc region may be modified to remove an ADCC site. ADCC sites are known in the art; see, for example, Molec. Immunol. 29 (5): 633-9 (1992) with regard to ADCC sites in IgG1. Specific examples of variant Fc domains are disclosed for example, in WO 97/34631 and WO 96/32478.

### Antibody-Cytokine Fusion Proteins

Immunocytokines (antibody-cytokine fusion proteins) represent an effective approach to target the immunosuppressive tumor microenvironment (TME) through the specific interaction between the antibody portion and the tumor antigens. Once bound to the target tumor, the carried cytokine such as interleukin-2 (IL-2) of immunocytokines (composed of either full antibody or single chain Fv conjugated to IL-2) can promote the in situ recruitment and activation of natural killer (NK) cells and cytotoxic CD8+ T lymphocytes (CTL). This recruitment induces a TME switch toward a classical T helper 1 (Th1) anti-tumor immune response. The modulation of the TME can be also achieved with immunocytokines with a mutated form of IL-2 that impairs regulatory T (Treg) cell proliferation and activity. Preclinical animal models and more recently phase I/II clinical trials have shown that IL-2 immunocytokines can avoid the severe toxicities of high doses of soluble IL-2. Also, very promising results have been reported using IL-2 immunocytokines in combination with other immunocytokines, chemo-, radio-, anti-angiogenic therapies, and blockade of immune checkpoints. However, fusion of a cytokine with an antibody has greatly reduced both cytokine activity (about 20 fold lower) and antibody circulation half-life (reduced to a few hours).

This invention will address those problems by providing not only a synergized benefits of anti-CD3/anti-PD-L1 with IL-2 and/or IL-10 to stimulate immune response to fight against cancer while reducing IL-2 and/or IL-10 side effects as observed in administration of IL-2 and IL-10 separately, but also the enhanced drug circulation half-life with PEGylation technology.

### Novel Design Providing Reduced Toxicity and Boosted Anti-Tumor Activity

Clinical development of anti-CD3 monoclonal antibody approved by FDA in 1985 was a symbol of modern antibody therapy.

Activation of T cells with anti-CD3 antibody and T cell expansion with IL-2 have been widely used in adoptive T cell transfer therapy and resulted in benefits to some patients of melanoma. However its severer side effects have greatly limited its clinical use. T cell activation is triggered with the binding of its CD3 receptor with a ligand or antibody. Because of universal presence of CD3 component in TCR complex in T cells, binding of anti-CD3 antibody with CD3 receptor has caused sometime severe side effects with patients.

Many efforts have been put to reduce toxicity of immune check point monoclonal antibodies such as CTLA-4, PD-L1 etc. One example is Probody technology (U.S. Pat. No. 8,563,269B2) which is designed to cap the active site of the protein drug during circulation and to remove the cap to reactivate the protein drug at the tumor site by the proteases that exist at significantly elevated level in tumor microenvironment. With this approach, the protein drug molecules are masked and toxicity to healthy tissue is reduced. However, the inert cap in the Probody technology provides no extra therapeutic value other than just providing the capping functionality.

This invention provides a novel structure format of PEGylated bispecific antibody capped with therapeutic cytokines that are not only providing reduced toxicity desired, but also boosting anti-tumor immunity of the drugs. With this invention, all the problems and shortcomings listed above could be addressed. For example, by adding cytokine caps such as IL2, IL10 etc. to the binding sites of the bispecific antibodies (that is to the anti-CD3 and/or anti-PDL1 of the anti-CD3/anti-PDL1 bispecific antibody), one could protect the PD-L1 expressing healthy tissue cells (including activated T cells) from being destroyed by the potent cytotoxic effect of anti-CD3/anti-PDL1 bispecific antibody during circulation. When IL2 and/or IL10 capped anti-CD3/anti-PDL1 bispecific antibody arrive tumor sites, the cytokine caps could be cleaved off from the bispecific antibody by the enzymes that are significantly elevated on the tumor sites. Examples of such enzymes include MMPs and uPA. The released naked anti-CD3/anti-PDL1 bispecific antibody would restore the binding affinity of the bispecific molecules while the cytokines IL2 and/or IL10 released on the tumor sites could enhance the anti-tumor immunity. Accordingly, this invention addresses the above discussed problems and improves cancer immunotherapy with the novel bispecific antibody technology.

### Compound

In one aspect of the invention, a compounds of formula (Ia) is provided.

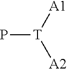

P is a non-immunogenic polymer. T is a multi-functional moiety, such as a trifunctional small molecule derived linker moiety, and may have two or more functional groups that are capable of site-specific conjugation with two different proteins. A1 and A2 can be any two different proteins, such as cytokine capped antibody fragments or cytokine capped single chain antibodies or other forms of cytokine capped antibodies or any combination of such.

In particular, an aspect of the invention provides a compound of Formula Ib:

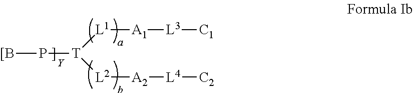

C₁ and C₂ are immunostimulant cytokine cap. C₁ could be the same as C₂ or could be different from C₂. L³ and L⁴ are cleavable enzymatic substrates.

The functional groups (e.g., two site-specific conjugation functional groups) that form linkages within (L¹)ₐ or (L²)_{b} or between (L¹)ₐ and protein A¹ or between (L²)_{b} and protein A² are selected from amine, thiol, maleimide, azide, alkyne, Dibenzocyclooctyl (DBCO), trans-cyclooctenes, tetrazines, carbonyl, hydrazide, oxime, triarylphosphine, potassium acyltrifluoroborates, and O-carbamoylhydroxylamines. The functional group can be placed in T or its adjacent component (L¹, L², A₁ or A₂).

a and b can each be an integer selected from 0-10, inclusive. In some embodiments, a and b are each 0.

L¹ and L² may each contain a spacer independently selected from the group consisting of —(CH₂)ₘXY(CH₂)ₙ—, —X(CH₂)ₘO(CH₂CH₂O)ₚ(CH₂)ₙY—, —(CH₂)ₘX—Y(CH₂)ₙ—, (CH₂)ₘheterocyclyl-, —(CH₂)ₘX—, and —X(CH₂)ₘY—, wherein m, n, and p in each instance are independently an integer ranging from 0 to 25; X and Y in each instance are independently selected from the group consisting of C(═O), CR₁R₂, NR₃, S, O, or Null; wherein R₁ and R₂ independently represent hydrogen, C₁₋₁₀ alkyl or (CH₂)₁₋₁₀C(═O), R₃ is H or a C₁₋₁₀ alkyl, and wherein the heterocyclyl is derived from an maleimido or a haloacetyl moiety.

The heterocyclyl moiety within linker L¹ and L² (whether it is at internal position or at terminal position) may be derived from a maleimido-based moiety. Non-limiting examples of suitable precursors include N-succinimidyl 4-(maleimidomethyl)cyclohexanecarboxylate (SMCC), N-succinimidyl-4-(N-maleimidomethyl)-cyclohexane-1-carboxy-(6-amidocaproate) (LC-SMCC), κ-maleimidoundecanoic acid N-succinimidyl ester (KMUA), γ-maleimidobutyric acid N-succinimidyl ester (GMBS), ε-maleimidcaproic acid N-hydroxysuccinimide ester (EMCS), m-maleimidobenzoyl-N-hydroxysuccinimide ester (MBS), N-(α-maleimidoacetoxy)-succinimide ester (AMAS), succinimidyl-6-(β-maleimidopropionamido)hexanoate (SMPH), N-succinimidyl 4-(p-maleimidophenyl)-butyrate (SMPB), and N-(p-maleimidophenyl)isocyanate (PMPI).

In some other non-limiting exemplary embodiments, the heterocyclyl moiety within linker L¹ and L² is derived from a haloacetyl-based moiety selected from N-succinimidyl-4-(iodoacetyl)-aminobenzoate (STAB), N-succinimidyl iodoacetate (SIA), N-succinimidyl bromoacetate (SBA), or N-succinimidyl 3-(bromoacetamido)propionate (SBAP).

In some embodiments, each of (Oₐ and (L²)_{b} may comprise:

X¹—(CH₂)ₘC(O)NR¹(CH₂)ₙO(CH₂CH₂O)ₚ(CH₂)_{q}C(O)— or

X³—(CH₂)ₘC(O)NR¹(CH₂)ₙO(CH₂CH₂O)ₚ(CH₂)_{q}X² (CH₂)ᵣNR²—,

wherein X¹, X² and X³ may be the same or different and independently represent a heterocyclyl group;

m, n, p, q, and r are each an integer selected from 0, 1, 2, 3, 4, 5, 6, 7, 8, 9, 10, 11, 12, 13, 14, 15, 16, 17, 18, 19, 20, 21, 22, 23, 24, 25; and

R¹ and R² independently represent hydrogen or C₁₋₁₀ alkyl.

In some embodiments, X¹ and/or X³ is derived from a maleimido-based moiety. In some embodiments, X² represents a triazolyl or a tetrazolyl group. In some embodiments, R¹ and R² each represent a hydrogen. In some embodiments, m, n, p, q, and r are each independently selected from 0, 1, 2, 3, 4, 5, 6, 7, 8, 9, and 10.

In some embodiments, the heterocyclyl linkage group of the linker may be derived from the reaction between tetrazole and alkene or between alkyne and azide. Thus, the heterocyclyl group can serve as a linkage point.

In some embodiments, (L¹)ₐ, (L²)_{b} and T are independently a peptide linker derived from no more than 25 amino acids.

In other embodiments, T is derived from Cysteine, Lysine, Asparagine, Aspartic, Glutamic acid, Glutamine, Histidine, Serine, Threonine, Tryptophan, Tyrosine or any unnatural amino acid such as genetically-encoded amino acid with alkene handle, para-acetyl-phenylalanine and etc. For instance, two functional groups of an amino acid can form linkages with (L¹)ₐ and (L²)_{b}, or with A₁ and A₂, and a third functional group forms the linkage with P. In further example, the amino and carboxylic acid of cysteine can form linkages with (L¹)ₐ and (L²)_{b}, or with A₁ and A₂. Meanwhile, the sulfur of cysteine forms a linkage with P.

C₁ and C₂ can be the same or different. In some embodiments, C₁ and C₂ can are selected independently from the group consisting of IL-2, IL-4, IL-10, IL-12, IL-15, and interferon-gamma. In some embodiments, one of C₁ and C₂ is IL2v and the other is IL10.

L³ or L⁴ are each a substrate of a cleavage enzyme. In some embodiments, L³ or L⁴ are each a substrate of a protease selected from the group consisting of a collagenase, a gelatinases, a matrilysin, MMP-12, a membrane type MMP, a stromelysin, a MMP-21, a MMP-27, a disintegrin, a metalloproteina with thrombospondin motif (ADAMTs), a procathepsin B, a cathepsin B, a cathepsin S, a caspase, a chondroitinase, a Hyaluronidase, uPA, and tPA.

The cleavage enzymes can be collagenases (e g., MMP-1, MMP-8, MMP-13), Gelatinases (e.g., gelatinase A, gelatinase B), matrilysins (e.g., matrilysin-1, matrilysin-2), MMP-12, membrane type MMPs (e.g., MMT-14, MMP-15, MMP-16, MMP-17, MMP-24, MMP-25), Stromelysins (e.g., Stromelysin-1/MMP_3, Stromelysin-2/MMP-10, Stromelysin-3/MMP-11), MMP-21, MMP-27, a disintegrin and metalloproteinase with thrombospondin motifs (ADAMTs) (e.g., ADAMTS-1, ADAMTS-2 ADAMTS-3, ADAMTS-4, ADAMTS-5, ADAMTS-6, ADAMTS-8, ADAMTS-9, ADAMTS-12, ADAMTS-13, ADAMTS-17, ADAMTS-18), Procathepsin B, cathepsin B, cathepsin S, cathepsin B, caspases (e.g., caspase 1, caspase 2, caspase 3, caspase 4, caspase 5, caspase 6, caspase 7, caspase 8, caspase 9, caspase 1,0 caspase 11, caspase 12, caspase 13), Chondroitinase, Hyaluronidase, uPA, tPA, etc. Substrates of these exemplary proteases are:

Substrate peptide sequences

PLNL
PLNG(N)LVG
PAG(A)LYG
PLGL
PAGLVGPPN
PAG(E)LI(VL)G
PAELIGP
W(Y)EXDX
V(L)DEXDX
DEXDX
W(Y)EXDX
VEXDX
DEXDX
I(L)EXDX
AASLKG
AAALTS
IPRX
SGRSA

X in the above table can be any amino acid.

L³ could be the same as L⁴ or could be different from L⁴. Either C₁ and C₂ or L³ and L⁴ could be null. A₁ and A₂ can be single-chain antibody fragment.

In another aspect, during circulation the antigen binding activity of A₁ and/or A₂ are blocked by the capping cytokine C₁ and/or C₂. In further aspect, the activities of A₁ and/or A₂ are regained when the L³ and/or L⁴ are cleaved at tumor sites in the presence of proteases in extracellular matrix. In still another aspect, the released cytokine caps C₁ and/or C₂ at the tumor site can further stimulate T cells proliferations and synergize cancer killing power.

In one aspect of this invention, methods of preparing two different anus of PEGylated bispecific antibody with cytokine cap are provided. In another aspect of the invention, methods of preparing terminal branched heterobifunctional PEG that is capable of site-specific conjugating with two different antibody fragments or single chain antibodies with cytokine caps are provided. In further aspect, methods for preparing PEGylated bispecific single chain antibody with cytokine caps thereof that is able to extend blood circulation half-life are also provided.

To synthesize PEGylated single chain bispecific antibody with cytokine cap, DNAs of two arms of single-chain bispecific antibody with cytokine caps can be synthesized and cloned separately in vitro and are introduced into, e.g., the CHO expression systems. Two proteins can be expressed and purified as described previously (WO2018075308). A terminal functional group of PEG such as hydroxyl or carboxyl group etc., can be activated and conjugated with a trifunctional small molecule moiety such as Boc protected lysine to form a terminal branched heterobifunctional PEG. The newly formed carboxyl group can be then converted to alkyne group by coupling with a small molecule spacer that has alkyne group. The amino group after Boc deprotection of the resulting compound can be conjugated with another small molecule spacer that has maleimide group to form a terminal branched maleimide/alkyne heterobifunctional PEG. The resulting maleimide/alkyne terminal branched heterobifunctional PEG is site-specifically conjugated with a thiol tagged single chain antibody with or without cytokine cap and an azide tagged single chain antibody with or without cytokine cap consecutively to form a PEGylated single chain bispecific antibody with cytokine caps, which provides longer blood circulation half-life, less toxicity to the health tissue and boosted anti-tumor activity.

### Non-Immunogenic Polymer and Trifunctional Linker T

The non-immunogenic polymer can be selected from polyethylene glycol (PEG), dextrans, carbohydrate-base polymers, polyalkylene oxide, polyvinyl alcohols, hydroxypropyl-methacrylamide (HPMA), and a co-polymer thereof.

The polymer may be derived from a precursor having a terminal functional group selected from carboxylic acid, amine, thiol, haloacetyl-based moiety, a maleimido-based moiety, azide, alkyne, Dibenzocyclooctyl (DBCO), trans-cyclooctenes, tetrazines, carbonyl, hydrazide, oxime, triarylphosphine, potassium acyltrifluoroborates, and O-carbamoylhydroxylamines. In exemplary embodiments, the linkage of T to P is derived from a pair of functional groups selected from the group consisting of thiol and maleimide, haloacetyl, carboxylic acid and amine, azide and alkyne, trans-cyclooctene and tetrazine, carbonyl and hydrazide, carbonyl and oxime, azide and triarylphosphine, and potassium acyltrifluoroborates and O-carbamoylhydroxylamines.

The terminal functional group reacts with a functional group of a precursor of T and gives rise to linkage such as amide, ester, carbamate, ether, thioether, disulfide, and various other heterocyclyl group.

In some embodiments, the terminal functional group leads to a heterocyclyl linkage with T and is derived from a maleimido-based moiety. Non-limiting examples of suitable precursors include N-succinimidyl 4-(maleimidomethyl)cyclohexanecarboxylate (SMCC), N-succinimidyl-4-(N-maleimidomethyl)-cyclohexane-1-carboxy-(6-amidocaproate) (LC-SMCC), κ-maleimidoundecanoic acid N-succinimidyl ester (KMUA), γ-maleimidobutyric acid N-succinimidyl ester (GMBS), ε-maleimidcaproic acid N-hydroxysuccinimide ester (EMCS), m-maleimidobenzoyl-N-hydroxysuccinimide ester (MBS), N-(a-maleimidoacetoxy)-succinimide ester (AMAS), succinimidyl-6-(β-maleimidopropionamido)hexanoate (SMPH), N-succinimidyl 4-(p-maleimidophenyl)-butyrate (SMPB), and N-(p-maleimidophenyl)isocyanate (PMPI).

In some embodiments, the terminal functional group leads to a heterocyclyl linkage with T and is derived from a haloacetyl-based moiety such as N-succinimidyl-4-(iodoacetyl)-aminobenzoate (STAB), N-succinimidyl iodoacetate (SIA), N-succinimidyl bromoacetate (SBA), and N-succinimidyl 3-(bromoacetamido)propionate (SBAP).

In some embodiments, the non-immunogenic polymer contains PEG, which can be optionally capped with methyl or a C₁₋₁₀ alkyl group. In some embodiments, the PEG has a molecular weight ranging from 1000 to 200,000, from 1000 to 150,000, from 1000 to 100,000, from 2000 to 150,000, from 2000 to 100,000, from 3000 to 80,000, from 3000 to 50,000, from 3000 to 150,000, from 3000 to 100,000, from 5000 to 150,000, from 5000 to 100,000, from 5000 to 80,000, from 10,000 to 150,000, from 10,000 to 100,000, from 10,000 to 50,000, from 10,000 to 20,000, or from 10,000 to 15,000.

Various types PEG can be used for the compound herein. In some embodiments, the PEG is prepared as reported in WO2018075308, the entire disclosure of which is incorporated herein by reference. In some embodiments, the PEG is linear shaped. In some embodiments, the PEG is branch shaped. In some embodiments, the PEG has multiple amts.

In one embodiment of present invention, the PEG can be of the formula:

B—O—(CH₂CH₂O)ₙCH₂(CH₂)ₘF

In the formula, n can be an integer from about 10 to 2300 to preferably provide polymer having a total molecule weight of from 10000 to 40000 or greater if desired. B can be methyl or other low molecule weight alkyl group or —CH₂(CH₂)ₘF. Non-limiting examples of B include methyl, ethyl, isopropyl, propyl, and butyl. M can be from 0 to 10. F can be a terminal functional group such as hydroxyl, carboxyl, thiol, halide, amino group etc. which is capable of being functionalized, activated and/or conjugating a trifunctional small molecule compound.

In another embodiment of present invention, the method can also be carried out with an alternative branched PEG. The branched PEG can be of the formula:

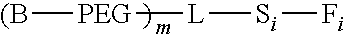

In this formula, PEG can be polyethylene glycol. M can be an integer greater than 1 to preferably provide polymer having a total molecule weight of from 10000 to 40000 or greater if desired. B can be methyl or other low molecule weight alkyl group. L can be a functional linkage moiety to that two or more PEGs are attached. Examples of such linkage moiety are: any amino acids such as glycine, alanine, Lysine, or 1,3-diamino-2-propanol, triethanolamine, any 5 or 6 member aromatic ring or aliphatic rings with more than two functional groups attached, etc. S is any non-cleavable spacer. F can be a terminal functional group such as hydroxyl, carboxyl, thiol, amino group, etc. i is 0 or 1. When i equals to 0, the formula become:

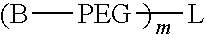

wherein: the definitions of PEG, m, B or L have the same foregoing meaning.

In some embodiments of the present invention, the multi-arm polymer moiety can be derived from a structure of the following formula.

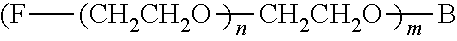

In this formula, n can be an integer and from about 10 to 1200 and m can bean integer and greater than 1 to preferably provide polymer having a total molecule weight of from 10000 to 40000 or greater if desired. F can be a terminal functional group such as hydroxyl, carboxyl, thiol, amino group, etc. B can be a non-functional linkage moiety to that two or more PEGs are attached. The structure of B can be symmetric or asymmetric, linear or cyclic saturated aliphatic group, and one or more carbons of B may be replaced with a heteroatoms such as oxygen, sulfur or nitrogen.

The method of the present invention can also be carried out with alternative polymeric substances such as dextrans, carbohydrate-base polymers, polyalkylene oxide, polyvinyl alcohols or other similar non-immunogenic polymers, the terminal groups of which are capable of being functionalized or activated to be converted to heterobifunctional groups. The foregoing list is merely illustrative and not intended to restrict the type of non-antigenic polymer suitable for use herein.

As described above, the trifunctional linker T can be derived from any suitable natural or non-natural amino acids. Non-limiting examples include Cysteine, Lysine, Asparagine, Aspartic, Glutamic acid, Glutamine, Histidine, Serine, Threonine, Tryptophan, Tyrosine, amino acid with alkene handle, para-acetyl-phenylalanine and analogs thereof.

In some embodiments, P is derived from a PEG having a terminal maleimide, T is derived from cysteine, and the linkage between P and T is a thioether. In some embodiments,

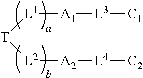

is derived from IL2v-SCACD3-L⁵-SCAPDL1-IL10, where T is connected to P via a cysteine thiol, wherein L⁵ is a peptide linker or null. In some embodiments, L⁵ has less than 25, less than 15, less than 10, or less than 5 amino acids. Various amino acids have been described above. In some embodiments,

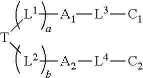

is IL2v-SCACD3-SCAPDL1-IL10, where T is connected to P via the thiol moiety of a cysteine.

### Chimeric Construct (Fusion Protein)

A related aspect provide a chimeric construct of Formula II,

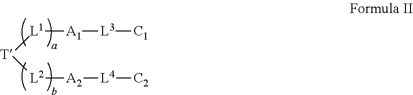

wherein:

A₁ and A₂ are two different antibodies, antibody fragments or single chain antibodies or other forms of antibodies or any combination thereof,

C₁ and C₂ are each a cleavable cap;

L³ and L⁴ are each an enzyme cleavable substrate or null;

L¹ and L² are each independently a bifunctional linker or a peptide linker;

a and b can each be an integer selected from 0-10, inclusive;

and

T′ is a linker moiety capable of forming two or three linkages, and one of the linkages can be with a polymer as described above. In some embodiments, T′ may contain a free functional group selected from carboxylic acid, amine, thiol, haloacetyl-based moiety, and maleimido-based moiety for forming a linkage with another structural component such as a polymer.

T′ is derived from a natural or unnatural amino acid as described. In exemplary embodiments, the carboxylic acid group and amino group of an amino acid form a linkage with L¹ and L², respectively (or with A¹ and A²). In some embodiments, it is derived from lysine, or cysteine. For instance, an amino group and a carboxylic group of lysine or cysteine can form a linkage with L¹ and L², respectively (or with A¹ and A²). Meanwhile, the free amino or thiol group T′ can react with the terminal functional group of a polymer.

In some embodiments, T′ is derived from cysteine, Lysine, Asparagine, Aspartic, Glutamic acid, Glutamine, Histidine, Serine, Threonine, Tryptophan, Tyrosine or genetically-encoded alkene lysines (such as N6-(hex-5-enoyl)-L-lysine), 2-Amino-8-oxononanoic acid, m or p-acetyl-phenylalanine, amino acid bearing a β-diketone side chain (such as 2-amino-3-(4-(3-oxobutanoyl)phenyl)propanoic acid), (S)-2-amino-6-(((1R,2R)-2-azidocyclopentyloxy)carbonylamino)hexanoic acid, azidohomoalanine, pyrrolysine analogue N⁶-((prop-2-yn-1-yloxy)carbonyl)-L-lysine, (S)-2-Amino-6-pent-4-ynamidohexanoic acid, (S)-2-Amino-6-((prop-2-ynyloxy)carbonylamino)hexanoic acid, (S)-2-Amino-6-((2-azidoethoxy)carbonylamino)hexanoic acid, p-azidophenylalanine, para-azidophenylalanine, N^{ε}-Acryloyl-1-lysine, NE-5-norbornene-2-yloxycarbonyl-1-lysine, N-ε-(Cyclooct-2-yn-1-yloxy)carbonyl)-L-lysine, N-ε-(2-(Cyclooct-2-yn-1-yloxy)ethyl) carbonyl-L-lysine, genetically Encoded Tetrazine Amino Acid (such as 4-(6-methyl-s-tetrazin-3-yl)aminophenylalanine) etc.

In some embodiments, C₁=IL2v, L³=uPA substrate or other protease substrate such as MMP14 or a combination of these protease substrates, A₁=SCACD3, A₂=SCAPDL1, L⁴=uPA substrate or other protease substrate such as MMP14 or a combination of these protease substrates, C₂=IL10, L¹ and/or L² are independently a peptide linker or null, and T′ is derived from an amino acid wherein the carboxylic acid group and amino group of the amino acid form a linkage with L¹ and L², respectively (or with A¹ and A²). In some embodiments, T′ is derived from lysine, or cysteine, wherein the amino group and the carboxylic group of lysine or cysteine form a linkage with L¹ and L², respectively (or with A¹ and A²). In some embodiments, a and b are each 0.

In some embodiments, Formula II is selected from IL2v-SCACD3-SCAPDL1-IL10, IL2v-MMP14-SCACD3-SCAPDL1-MM14-IL10, IL2v-uPA-SCACD3-SCAPDL1-uPA-IL10, and SCACD3-SCAPDL1.

In some embodiments, Formula II is IL2v-L³-SCACD3-L⁵-SCAPDL1-L⁴-IL10, wherein L⁵ is a peptide linker. In some embodiments, Formula II is IL2v-SCACD3-L⁵-SCAPDL1-IL10. In some embodiments, L⁵ has less than 25, less than 15, less than 10, or less than 5 amino acids.

### Synthesis

Once the desired PEG has been selected, the terminal functional group of PEG such as hydroxyl, carboxyl group etc. can be converted to terminal branched heterobifunctional groups using any art-recognized process. Broadly stated, the terminal branched heterobifunctional PEG such as terminal branched heterobifunctional maleimide/alkyne PEG can be prepared by activating terminal hydroxyl or carboxyl group of the PEG with N-Hydroxysuccinimide using reagents such as Di(N-succinimidyl) carbonate (DSC), triphosgene etc. in the case of terminal hydroxyl group or coupling reagents such as N,N′-Diisopropylcarbodiimide (DIPC), 1-Ethyl-3-(3-dimethylaminopropyl)carbodiimide (EDC) etc. in the case of terminal carboxyl group in the presence of base such as 4-Dimethylaminopyridine (DMAP), pyridine etc. to form activated PEG.

Next, the activated PEG can be reacted with a trifunctional small molecule such as lysine derivative H-Lys(Boc)-OH in the presence of base such as Diisopropylamine (DIPE) to form a terminal branched heterobifunctional PEG with a free carboxyl group and a Boc protected amino group. As will be appreciated by those of ordinary skill, other terminal functional groups of PEG such as halide, amino, thiol group etc. and other tri-functional small molecules containing any combination of three functional groups from the list of NH2, NHNH2, COOH, OH, C═OX, N═C═X, S, anhydride, halides, maleimid, C═C, C═C etc. or their protected version can be used as alternatives for the same purpose if desired.

The terminal branched carboxyl/Boc amino heterobifunctional PEG can be then converted to a terminal branched alkyne/Boc amino heterobifunctional PEG by coupling with a small molecule spacer that has alkyne group such as 1-amino-3-butyne. Treatment of a terminal branched alkyne/Boc amino heterobifunctional PEG with an acid such as trifluoroacetic acid (TFA) gives the terminal branched alkyne/amine heterobifunctional PEG. The target terminal branched alkyne/maleimide heterobifunctional PEG can be obtained by reacting the terminal branched alkyne/amine heterobifunctional PEG with another small molecule spacer that has a maleimide group such as NHS-PEG2-Maleimide. This terminal branched alkyne/maleimide heterobifunctional PEG is capable of site-specific conjugation with a thiol tagged antibody and an azide tagged antibody consecutively.

For synthesizing desired protein target, IL2V+uPA+MMP14 substrate+anti-CD3 (SCACD3)) and IL10+uPA+MMP14 substrate+anti-PDL1(SCAPDL1) can be made separately. Both proteins can be made via recombinant DNA technology in any suitable expression system, such as Chinese hamster ovary (CHO) cells with GS knock out using pD2531nt-HDP expression vector containing GS gene (both the cell line and the vector are licensed from Horizon Discovery, Inc). In one example, DNAs encoding the first protein (IL2v+uPA+MMP14 substrate+scFv of anti-CD3(SCACD3)) and the second protein (IL10+uPA+MMP14 substrate+scFv of anti-PDL1(SCAPDL1)) can be synthesized and cloned into pD2531nt-HDP expression vector and transfected to CHO-GS(−/−) cells. Stable cell lines with high production capacity can be obtained by cultured the cells in medium containing GS inhibitor MSX while not supplemented with glutamine. The two proteins produced by such cell lines can be purified by Ni-chelating resin. To facilitate the subsequent conjugation, a site-specific functional group such as thiol can be inserted through recombinant DNA technology into the linker between VH and VL of the single chain antibodies. Pure proteins can be obtained via chromatographic process. As will be appreciated by those of ordinary skill, other known site-specific functional groups can also be inserted through recombinant DNA technology into the linker between VH and VL of the SCA as alternatives for the same purpose if desired.

To prepare PEGylated single chain bispecific antibody with the caps, the terminal branched alkyne/maleimide heterobifunctional PEG can be reacted site specifically with free thiol functional group of capped SCACD3 that is genetically inserted, resulting in PEG-(SCACD3)-DBCO, while capped SCAPDL1 is conjugated site specifically with a small molecule azide/maleimide bifunctional linker, resulting in capped azide-SCAPDL1. Purified capped azide-SCAPDL1 and capped PEG-(SCACD3)-DBCO can be reacted site specifically through an azide-DBCO clicking chemistry to form a capped PEGylated single chain bispecific antibody PEG-SCACD3/SCAPDL1/caps.

In addition to thiol/maleimide and azide/alkyne site specific conjugation group pair used in this invention, as will be appreciated by those of ordinary skill, other known pairs of site-specific conjugation groups, such as DBCO/azide pair; trans-cyclooctenes/tetrazines pair; carbonyl/hydrazide pair; carbonyl/oxime pair; azide/triarylphosphine pair; potassium acyltrifluoroborates/O-carbamoylhydroxylamines pair, can be similarly designed and used as alternatives for the same purpose if desired. The foregoing list of site-specific conjugation group pairs is merely illustrative and not intended to restrict the type of site-specific conjugation group pairs suitable for use herein.

An alternative approach to prepare the compounds disclosed herein include:

reacting

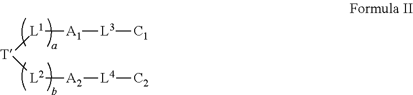

with a non-immunogenic polymer having a terminal functional group. The T′ moiety reacts with the terminal functional group of the polymer to form a linkage. The substituents of Formula II are as defined above.

The terminal functional group of the polymer is the same as described above. In some embodiments, the terminal functional group is selected from carboxylic acid, amine, thiol, haloacetyl-based moiety, and maleimido-based moiety.

The polymer may contain, as described above, polyethylene glycol (PEG), dextrans, carbohydrate-base polymers, polyalkylene oxide, polyvinyl alcohols, hydroxypropyl-methacrylamide (HPMA), and a co-polymer thereof. In some embodiments, the polymer is PEG, and the specific structure and functional group of the PEG is as described above.

T′ is derived from a natural or unnatural amino acid as described above except that T′ bears a free functional group for forming the linkage between T and P. In some embodiments, it is derived from lysine, or cysteine. For instance, an amino group and a carboxylic group of lysine or cysteine can form a linkage with L¹ and L², respectively (or with A¹ and A². Meanwhile, the free amino or thiol group T′ reacts with the terminal functional of P.

Specific reaction conditions for the synthesis of the compounds described herein can be identified by one of ordinary skill in the art without undue experiments in view of general knowledge of the field as described in literature references including Amino Acid and Peptide Synthesis, Oxford University Press; 2 edition (Aug. 1, 2002) and Practical Synthetic Organic Chemistry: Reactions, Principles, and Techniques, Wiley; 2 edition (Feb. 5, 2020), the entire disclosure of these references are hereby incorporated by reference.

### Compositions

The present invention also provides a composition, e.g., a pharmaceutical composition, containing the compound of the present invention, formulated together with a pharmaceutically acceptable carrier. For example, a pharmaceutical composition of the invention can comprise a compound that binds to both CD3 and PDL1.

Therapeutic formulations of this invention can be prepared by mixing the multi-specific molecules having the desired degree of purity with optional physiologically acceptable carriers, excipients or stabilizers (Remington's Pharmaceutical Sciences 16th edition, Osol, A. Ed. (1980)), in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN, PLURONICS, or polyethylene glycol (PEG).

The formulation may also contain more than one active compound as necessary for the particular indication being treated, preferably those with complementary activities that do not adversely affect each other. For instance, the formulation may further comprise another antibody, cytotoxic agent, or a chemotherapeutic agent. Such molecules are suitably present in combination in amounts that are effective for the purpose intended.

The active ingredients may also be entrapped in microcapsule prepared, for example, by coacervation techniques or by interfacial polymerization, for example, hydroxymethylcellulose or gelatin-microcapsule and poly-(methylmethacylate) microcapsule, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

Sustained-release preparations may be prepared. Suitable examples of sustained-release preparations include semipermeable matrices of solid hydrophobic polymers containing the multi-specific molecules, which matrices are in the form of shaped articles, e.g., films, or microcapsule. Examples of sustained-releasable matrices include polyesters, hydrogels (for example, poly(2-hydroxyethyl-methacrylate), or poly(vinylalcohol)), polylactides (U.S. Pat. No. 3,773,919), copolymers of L-glutamic acid and γ ethyl-L-glutamate, non-degradable ethylene-vinyl acetate, degradable lactic acid-glycolic acid copolymers such as the LUPRON DEPOT (injectable microspheres composed of lactic acid-glycolic acid copolymer and leuprolide acetate), and poly-D-(−−)-3-hydroxybutyric acid. While polymers such as ethylene-vinyl acetate and lactic acid-glycolic acid enable release of molecules for over 100 days, certain hydrogels release proteins for shorter time periods. When encapsulated antibodies remain in the body for a long time, they may denature or aggregate as a result of exposure to moisture at 37° C., resulting in a loss of biological activity and possible changes in immunogenicity. Rational strategies can be devised for stabilization depending on the mechanism involved. For example, if the aggregation mechanism is discovered to be intermolecular S—S bond formation through thio-disulfide interchange, stabilization may be achieved by modifying sulfhydryl residues, lyophilizing from acidic solutions, controlling moisture content, using appropriate additives, and developing specific polymer matrix compositions.

Pharmaceutical compositions of the invention can be administered in combination therapy, i.e., combined with other agents. Examples of therapeutic agents that can be used in combination therapy are described in greater detail below.

The formulations to be used for in vivo administration must be sterile. This can be readily accomplished by filtration through sterile filtration membranes. Sterile injectable solutions can be prepared by incorporating the active compound in the required amount in an appropriate solvent with one or a combination of ingredients enumerated above, as required, followed by sterilization microfiltration. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying (lyophilization) that yield a powder of the active ingredient plus any additional desired ingredient from a previously sterile-filtered solution thereof.

### Dosage

The amount of active ingredient which can be combined with a carrier material to produce a single dosage form will vary depending upon the subject being treated, and the particular mode of administration. The amount of active ingredient which can be combined with a carrier material to produce a single dosage form will generally be that amount of the composition which produces a therapeutic effect. Generally, out of one hundred percent, this amount will range from about 0.01 percent to about ninety-nine percent of active ingredient, preferably from about 0.1 percent to about 70 percent, most preferably from about 1 percent to about 30 percent of active ingredient in combination with a pharmaceutically acceptable carrier.

Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier. The specification for the dosage unit forms of the invention are dictated by and directly dependent on (a) the unique characteristics of the active compound and the particular therapeutic effect to be achieved, and (b) the limitations inherent in the art of compounding such an active compound for the treatment of sensitivity in individuals.

For administration of the multi-specific molecules of this invention, the dosage ranges from about 0.0001 to 100 mg/kg, and more usually 0.01 to 50 mg/kg, of the host body weight. For example dosages can be 0.3 mg/kg body weight, 1 mg/kg body weight, 3 mg/kg body weight, 5 mg/kg body weight or 10 mg/kg body weight or within the range of 1-10 mg/kg. An exemplary treatment regime entails administration twice per week, once per week, once every two weeks, once every three weeks, once every four weeks, once a month, once every 3 months or once every three to 6 months. Preferred dosage regimens for multi-specific molecules of the invention include 1 mg/kg body weight or 3 mg/kg body weight via intravenous administration, with the multi-specific molecule being given using one of the following dosing schedules: (i) every four weeks for six dosages, then every three months; (ii) every three weeks; (iii) 3 mg/kg body weight once followed by 1 mg/kg body weight every three weeks.

Alternatively, multi-specific molecules can be administered as a sustained release formulation, in which case less frequent administration is required. Dosage and frequency vary depending on the half-life of the multi-specific molecules in the patient. In general, human antibodies show the longest half-life, followed by humanized antibodies, chimeric antibodies, and nonhuman antibodies. The dosage and frequency of administration can vary depending on whether the treatment is prophylactic or therapeutic. In prophylactic applications, a relatively low dosage is administered at relatively infrequent intervals over a long period of time. Some patients continue to receive treatment for the rest of their lives. In therapeutic applications, a relatively high dosage at relatively short intervals is sometimes required until progression of the disease is reduced or terminated, and preferably until the patient shows partial or complete amelioration of symptoms of disease. Thereafter, the patient can be administered a prophylactic regime.

Actual dosage levels of the active ingredients in the pharmaceutical compositions of the present invention may be varied so as to obtain an amount of the active ingredient which is effective to achieve the desired therapeutic response for a particular patient, composition, and mode of administration, without being toxic to the patient. The selected dosage level will depend upon a variety of pharmacokinetic factors including the activity of the particular compositions of the present invention employed, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts.

A “therapeutically effective dosage” of a multi-specific molecule of the invention preferably results in a decrease in severity of disease symptoms, an increase in frequency and duration of disease symptom-free periods, or a prevention of impairment or disability due to the disease affliction. For example, for the treatment of tumors, a “therapeutically effective dosage” preferably inhibits cell growth or tumor growth or metastasis by at least about 20%, more preferably by at least about 40%, even more preferably by at least about 60%, and still more preferably by at least about 80% relative to untreated subjects. The ability of an agent or compound to inhibit tumor growth can be evaluated in an animal model system predictive of efficacy in human tumors. Alternatively, this property of a composition can be evaluated by examining the ability of the compound to inhibit, such inhibition in vitro by assays known to the skilled practitioner. A therapeutically effective amount of a therapeutic compound can decrease tumor size, metastasis, or otherwise ameliorate symptoms in a subject. One of ordinary skill in the art would be able to determine such amounts based on such factors as the subject's size, the severity of the subject's symptoms, and the particular composition or route of administration selected.

### Administration

A composition of the invention can be administered via one or more routes of administration using one or more of a variety of methods known in the art. As will be appreciated by the skilled artisan, the route and/or mode of administration will vary depending upon the desired results. Preferred routes of administration for antibodies of the invention include intravenous, intramuscular, intradermal, intraperitoneal, subcutaneous, spinal or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion. Alternatively, a multi-specific molecule of the invention can be administered via a non-parenteral route, such as a topical, epidermal or mucosal route of administration, for example, intranasally, orally, vaginally, rectally, sublingually or topically.

The active compounds can be prepared with carriers that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See, e.g., Sustained and Controlled Release Drug Delivery Systems, J. R. Robinson, ed., Marcel Dekker, Inc., New York, 1978.

Therapeutic compositions can be administered with medical devices known in the art. For example, a therapeutic composition of the invention can be administered with a needleless hypodermic injection device, such as the devices disclosed in U.S. Pat. Nos. 5,399,163, 5,383,851, 5,312,335, 5,064,413, 4,941,880, 4,790,824, and 4,596,556. Examples of well-known implants and modules useful in the present invention include those described in U.S. Pat. Nos. 4,487,603, 4,486,194, 4,447,233, 4,447,224, 4,439,196, and 4,475,196. These patents are incorporated herein by reference. Many other such implants, delivery systems, and modules are known to those skilled in the art.

### Treatment Methods

In one aspect, the present invention relates to treatment of a subject in vivo using the above-described multi-specific molecule such that growth and/or metastasis of cancerous tumors is inhibited. In one embodiment, the invention provides a method of inhibiting growth and/or restricting metastatic spread of tumor cells in a subject, comprising administering to the subject a therapeutically effective amount of a multi-specific molecule.

Non-limiting examples of preferred cancers for treatment include chronic or acute leukemia including acute myeloid leukemia, chronic myeloid leukemia, acute lymphoblastic leukemia, chronic lymphocytic leukemia, lymphocytic lymphoma, breast cancer, ovarian cancer, melanoma (e.g., metastatic malignant melanoma), renal cancer (e.g., clear cell carcinoma), prostate cancer (e.g., hormone refractory prostate adenocarcinoma), colon cancer and lung cancer (e.g., non-small cell lung cancer). Additionally, the invention includes refractory or recurrent malignancies whose growth may be inhibited using the antibodies of the invention. Examples of other cancers that may be treated using the methods of the invention include bone cancer, pancreatic cancer, skin cancer, cancer of the head or neck, cutaneous or intraocular malignant melanoma, uterine cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, Hodgkin's Disease, non-Hodgkin's lymphoma, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, solid tumors of childhood, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T-cell lymphoma, environmentally induced cancers including those induced by asbestos, and combinations of said cancers.

As used herein, the term “subject” is intended to include human and non-human animals. Non-human animals includes all vertebrates, e.g., mammals and non-mammals, such as non-human primates, sheep, dogs, cats, cows, horses, chickens, amphibians, and reptiles, although mammals are preferred, such as non-human primates, sheep, dogs, cats, cows and horses. Preferred subjects include human patients in need of enhancement of an immune response. The methods are particularly suitable for treating human patients having a disorder that can be treated by augmenting the immune response.

The above treatment may also be combined with standard cancer treatments. For example, it may be effectively combined with chemotherapeutic regimes. In these instances, it may be possible to reduce the dose of chemotherapeutic reagent administered (Mokyr, M. et al. (1998) Cancer Research 58: 5301-5304).

Other antibodies which may be used to activate host immune responsiveness can be used in or with the multi-specific molecule of this invention. These include molecules targeting on the surface of dendritic cells which activate DC function and antigen presentation. For example, anti-CD40 antibodies are able to substitute effectively for T cell helper activity and can be used in conjunction with the multi-specific molecule of this invention. Similarly, antibodies targeting T cell costimulatory molecules such as CTLA-4, OX-40, and ICOS or antibodies targeting PD-1 (U.S. Pat. No. 8,008,449) PD-1L (U.S. Pat. Nos. 7,943,743 and 8,168,179) may also provide for increased levels of T cell activation. In another example, the multi-specific molecule of this invention can be used in conjunction with anti-neoplastic antibodies, such as RITUXAN (rituximab), HERCEPTIN (trastuzumab), BEXXAR (tositumomab), ZEVALIN (ibritumomab), CAMPATH (alemtuzumab), LYMPHOCIDE (eprtuzumab), AVASTIN (bevacizumab), and TARCEVA (erlotinib), and the like.

### Definitions

The term “alkyl” as used herein refers to a hydrocarbon chain, typically ranging from about 1 to 25 atoms in length. Such hydrocarbon chains are preferably but not necessarily saturated and may be branched or straight chain, although typically straight chain is preferred. The term C1-10 alkyl includes alkyl groups with 1, 2, 3, 4, 5, 6, 7, 8, 9 and 10 carbons. Similarly C1-25 alkyl includes all alkyls with 1 to 25 carbons. Exemplary alkyl groups include methyl, ethyl, isopropyl, n-butyl, n-pentyl, 2-methyl-1-butyl, 3-pentyl, 3-methyl-3-pentyl, and the like. As used herein, “alkyl” includes cycloalkyl when three or more carbon atoms are referenced. Unless otherwise noted, an alkyl can be substituted or un-substituted.

The term “functional group” as used herein refers to a group that may be used, under normal conditions of organic synthesis, to form a covalent linkage between the entity to which it is attached and another entity, which typically bears a further functional group. A “bifunctional linker” refers to a linker with two functional groups forms two linkages via with other moieties of a conjugate.

The term “aryl” refers to a monovalent or divalent aromatic hydrocarbon group derived by the removal of one hydrogen atom from a single carbon atom of a parent aromatic ring system. Typical aryl groups include, but are not limited to, groups derived from aceanthrylene, acephenanthrylene, anthracene, azulene, benzene, fluoranthene, fluorene, hexacene, hexaphene, hexylene, as-indacene, s-indacene, indane, indene, naphthalene, octacene, octaphene, octalene, ovalene, penta-2,4-diene, pentacene, pentalene, pentaphene, perylene, phenalene, phenyl, phenanthrene, picene, and the like. Particularly, an aryl group comprises from 6 to 14 carbon atoms.

The term “derivative” as used herein refers to a chemically-modified compound with an additional structural moiety for the purpose of introducing new functional group or tuning the properties of the original compound.

The term “protecting group” as used herein refers to a moiety that prevents or blocks reaction of a particular chemically reactive functional group in a molecule under certain reaction conditions. Various protecting groups are well-known in the art and are described, for example, in T. W. Greene and G. M. Wuts, Protecting Groups in Organic Synthesis, Third Edition, Wiley, New York, 1999, and in P. J. Kocienski, Protecting Groups, Third Ed Thieme Chemistry, 2003, and references cited therein.

The term “PEG” or “poly(ethylene glycol)” as used herein refers to poly(ethylene oxide). PEGs for use in the present invention typically comprise a structure of —(CH2CH2O)n-. PEGs may have a variety of molecular weights, structures or geometries. A PEG group may comprise a capping group that does not readily undergo chemical transformation under typical synthetic reaction conditions. Examples of capping groups include —OC1-25 alkyl or —OAryl.

The term “linker” as used herein refers to an atom or a collection of atoms used to link interconnecting moieties, such as an antibody and a polymer moiety. A linker can be cleavable or noncleavable. The preparation of various linkers for conjugates have been described in literatures including for example Goldmacher et al., Antibody-drug Conjugates and Immunotoxins: From Pre-clinical Development to Therapeutic Applications, Chapter 7, in Linker Technology and Impact of Linker Design on ADC properties, Edited by Phillips G L; Ed. Springer Science and Business Media, New York (2013). Cleavable linkers incorporate groups or moieties that can be cleaved under certain biological or chemical conditions. Examples include enzymatically cleavable disulfide linkers, 1,4- or 1,6-benzyl elimination, trimethyl lock system, bicine-based self cleavable system, acid-labile silyl ether linkers and other photo-labile linkers.

The term “linking group” or “linkage group” or “linage” as used herein refers to a functional group or moiety connecting different moieties of a compound or conjugate. Examples of a linking group include, but are not limited to, amide, ester, carbamate, ether, thioether, disulfide, hydrazone, oxime, and semicarbazide, carbodiimide, acid labile group, photolabile group, peptidase labile group and esterase labile group. For example, a linker moiety and a polymer moiety may be connected to each other via an amide or carbamate linkage group.

The term “multiple arms” or “multi-armed” as used herein refers to the geometry or overall structure of a polymer refers to polymer having 2 or more polymer-containing “arms” connected to a “core” molecule or structure. Thus, a multi-armed polymer may possess 2, 3, 4, 5, 6, 7, 8 arms or more.

The terms “peptide,” “polypeptide,” and “protein” are used herein interchangeably to describe the arrangement of amino acid residues in a polymer. A peptide, polypeptide, or protein can be composed of the standard 20 naturally occurring amino acid, in addition to rare amino acids and synthetic amino acid analogs. They can be any chain of amino acids, regardless of length or post-translational modification (for example, glycosylation or phosphorylation).

A “recombinant” peptide, polypeptide, or protein refers to a peptide, polypeptide, or protein produced by recombinant DNA techniques; i.e., produced from cells transformed by an exogenous DNA construct encoding the desired peptide. A “synthetic” peptide, polypeptide, or protein refers to a peptide, polypeptide, or protein prepared by chemical synthesis. The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Within the scope of this invention are fusion proteins containing one or more of the afore-mentioned sequences and a heterologous sequence. A heterologous polypeptide, nucleic acid, or gene is one that originates from a foreign species, or, if from the same species, is substantially modified from its original form. Two fused domains or sequences are heterologous to each other if they are not adjacent to each other in a naturally occurring protein or nucleic acid.

An “isolated” peptide, polypeptide, or protein refers to a peptide, polypeptide, or protein that has been separated from other proteins, lipids, and nucleic acids with which it is naturally associated. The polypeptide/protein can constitute at least 10% (i.e., any percentage between 10% and 100%, e.g., 20%, 30%, 40%, 50%, 60%, 70%, 80%, 85%, 90%, 95%, and 99%) by dry weight of the purified preparation. Purity can be measured by any appropriate standard method, for example, by column chromatography, polyacrylamide gel electrophoresis, or HPLC analysis. An isolated polypeptide/protein described in the invention can be purified from a natural source, produced by recombinant DNA techniques, or by chemical methods.

An “antigen” refers to a substance that elicits an immunological reaction or binds to the products of that reaction. The term “epitope” refers to the region of an antigen to which an antibody or T cell binds.

The term “antibody” as referred to herein includes whole antibodies and any antigen binding fragment or single chains thereof. Whole antibodies are glycoproteins comprising at least two heavy (H) chains and two light (L) chains inter-connected by disulfide bonds. Each heavy chain is comprised of a heavy chain variable region (abbreviated herein as V_{H}) and a heavy chain constant region. The heavy chain constant region is comprised of three domains, C_{H}1, C_{H}2 and C_{H}3. Each light chain is comprised of a light chain variable region (abbreviated herein as V_{L}) and a light chain constant region. The light chain constant region is comprised of one domain, C_{L}. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. The heavy chain variable region CDRs and FRs are HFR1, HCDR1, HFR2, HCDR2, HFR3, HCDR3, HFR4. The light chain variable region CDRs and FRs are LFR1, LCDR1, LFR2, LCDR2, LFR3, LCDR3, LFR4. The variable regions of the heavy and light chains contain a binding domain that interacts with an antigen. The constant regions of the antibodies can mediate the binding of the immunoglobulin to host tissues or factors, including various cells of the immune system (e.g., effector cells) and the first component (CIq) of the classical complement system.

As used herein, “antibody fragments”, may comprise a portion of an intact antibody, generally including the antigen binding and/or variable region of the intact antibody and/or the Fc region of an antibody which retains FcR binding capability. Examples of antibody fragments include linear antibodies; single-chain antibody molecules; and multispecific antibodies formed from antibody fragments. Preferably, the antibody fragments retain the entire constant region of an IgG heavy chain, and include an IgG light chain.

The term “antigen-binding fragment or portion” of an antibody (or simply “antibody fragment or portion”), as used herein, refers to one or more fragments of an antibody that retain the ability to specifically bind to an antigen. It has been shown that the antigen-binding function of an antibody can be performed by fragments of a full-length antibody. Examples of binding fragments encompassed within the term “antigen-binding fragment or portion” of an antibody include (i) a Fab fragment, a monovalent fragment consisting of the V_{L}, V_{H}, C_{L} and C_{H}I domains; (ii) a F(ab′)2 fragment, a bivalent fragment comprising two Fab fragments linked by a disulfide bridge at the hinge region; (iii) a Fab′ fragment, which is essentially an Fab with part of the hinge region (see, FUNDAMENTAL IMMUNOLOGY (Paul ed., 3^{rd} ed. 1993)); (iv) a Fd fragment consisting of the V_{H} and C_{H}I domains; (v) a Fv fragment consisting of the V_{L} and VH domains of a single arm of an antibody, (vi) a dAb fragment (Ward et al., (1989) Nature 341:544-546), which consists of a VH domain; (vii) an isolated complementarity determining region (CDR); and (viii) a nanobody, a heavy chain variable region containing a single variable domain and two constant domains. Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding fragment or portion” of an antibody. These antibody fragments are obtained using conventional techniques known to those with skill in the art, and the fragments are screened for utility in the same manner as are intact antibodies.

As used herein, the term “Fc fragment” or “Fc region” is used to define a C-terminal region of an immunoglobulin heavy chain.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to conventional (polyclonal) antibody preparations that typically include different antibodies directed against different determinants (epitopes), each monoclonal antibody is directed against a single determinant on the antigen. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler and Milstein, Nature, 256, 495-497 (1975), which is incorporated herein by reference, or may be made by recombinant DNA methods (see, e.g., U.S. Pat. No. 4,816,567, which is incorporated herein by reference). The monoclonal antibodies may also be isolated from phage antibody libraries using the techniques described in Clackson et al., Nature, 352, 624-628 (1991) and Marks et al., J Mol Biol, 222, 581-597 (1991), for example, each of which is incorporated herein by reference.

The monoclonal antibodies herein specifically include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (see U.S. Pat. No. 4,816,567; Morrison et al., Proc Natl Acad Sci USA, 81, 6851-6855 (1984); Neuberger et al., Nature, 312, 604-608 (1984); Takeda et al., Nature, 314, 452-454 (1985); International Patent Application No. PCT/GB85/00392, each of which is incorporated herein by reference).

“Humanized” forms of non-human (e.g., murine) antibodies are chimeric antibodies that contain minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, Fv framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FR residues are those of a human immunoglobulin sequence. The humanized antibody optionally also will comprise at least a portion of an immunoglobulin constant region (Fc), typically that of a human immunoglobulin. For further details, see Jones et al., Nature, 321, 522-525 (1986); Riechmann et al., Nature, 332, 323-329 (1988); Presta, Curr Op Struct Biol, 2, 593-596 (1992); U.S. Pat. No. 5,225,539, each of which is incorporated herein by reference.

“Human antibodies” refer to any antibody with fully human sequences, such as might be obtained from a human hybridoma, human phage display library or transgenic mouse expressing human antibody sequences.

The term “pharmaceutical composition” refers to the combination of an active agent with a carrier, inert or active, making the composition especially suitable for diagnostic or therapeutic use in vivo or ex vivo.

As used herein, “pharmaceutically acceptable carrier” includes any and all solvents, dispersion media, coatings, antibacterial and antifungal agents, isotonic and absorption delaying agents, and the like that are physiologically compatible. A “pharmaceutically acceptable carrier,” after administered to or upon a subject, does not cause undesirable physiological effects. The carrier in the pharmaceutical composition must be “acceptable” also in the sense that it is compatible with the active ingredient and can be capable of stabilizing it. One or more solubilizing agents can be utilized as pharmaceutical carriers for delivery of an active agent. Examples of a pharmaceutically acceptable carrier include, but are not limited to, biocompatible vehicles, adjuvants, additives, and diluents to achieve a composition usable as a dosage form. Examples of other carriers include colloidal silicon oxide, magnesium stearate, cellulose, and sodium lauryl sulfate. Additional suitable pharmaceutical carriers and diluents, as well as pharmaceutical necessities for their use, are described in Remington's Pharmaceutical Sciences. Preferably, the carrier is suitable for intravenous, intramuscular, subcutaneous, parenteral, spinal or epidermal administration (e.g., by injection or infusion). The therapeutic compounds may include one or more pharmaceutically acceptable salts. A “pharmaceutically acceptable salt” refers to a salt that retains the desired biological activity of the parent compound and does not impart any undesired toxicological effects (see e.g., Berge, S. M., et al. (1977) J. Pharm. Sci. 66:1-19).

As used herein, “treating” or “treatment” refers to administration of a compound or agent to a subject who has a disorder or is at risk of developing the disorder with the purpose to cure, alleviate, relieve, remedy, delay the onset of, prevent, or ameliorate the disorder, the symptom of the disorder, the disease state secondary to the disorder, or the predisposition toward the disorder.

An “effective amount” refers to the amount of an active compound/agent that is required to confer a therapeutic effect on a treated subject. Effective doses will vary, as recognized by those skilled in the art, depending on the types of conditions treated, route of administration, excipient usage, and the possibility of co-usage with other therapeutic treatment. A therapeutically effective amount of a combination to treat a neoplastic condition is an amount that will cause, for example, a reduction in tumor size, a reduction in the number of tumor foci, or slow the growth of a tumor, as compared to untreated animals.

As disclosed herein, a number of ranges of values are provided. It is understood that each intervening value, to the tenth of the unit of the lower limit, unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither, or both limits are included in the smaller ranges is also encompassed within the invention, subject to any specifically excluded limit in the stated range. Where the stated range includes one or both of the limits, ranges excluding either or both of those included limits are also included in the invention.

The term “about” generally refers to plus or minus 10% of the indicated number. For example, “about 10%” may indicate a range of 9% to 11%, and “about 1” may mean from 0.9-1.1. Other meanings of “about” may be apparent from the context, such as rounding off, so, for example “about 1” may also mean from 0.5 to 1.4.

### EXAMPLES

The following examples serve to provide further appreciation of the invention but are not meant by any way to restrict the effective scope of the invention.

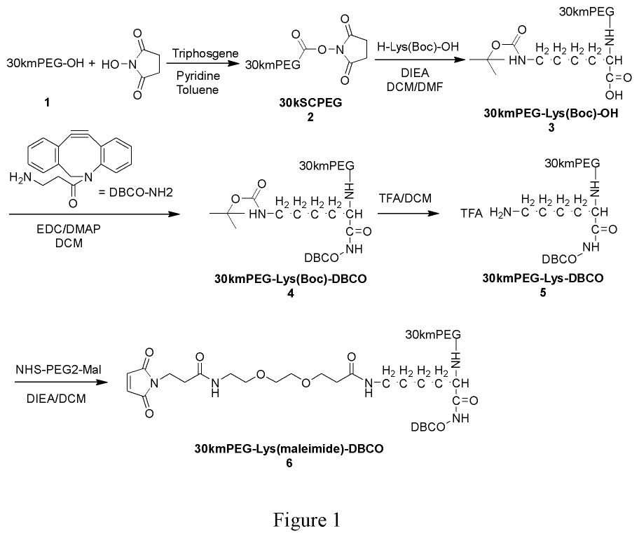

### Example 1. Preparation of 30kmPEG-Lys(Maleimide)-DBCO (FIG. 1)

Preparation of 30kmSC-PEG (compound 2):

25 g of 30kmPEG-OH (MW=30000, 1 eq) was azeotroped for two hours with 360 mL of reagent toluene to remove 75 mL toluene/water. After azeotroping, the solution was cooled to 45-50° C. 166 mg of triphosgene (0.67 eq.) was added to PEG followed by 131.8 mg of anhydrous pyridine (2 eq.). Reaction was stirred at 50° C. for 3 hours. 239.8 mg of N-hydroxysuccinimide (2.5 eq.) was then added followed by 164.8 g of anhydrous pyridine (2.5 eq.). The reaction mixture was stirred at 50° C. overnight under nitrogen. Pyridine salt was filtered. Solvent was removed with Rotavapor and the residue was recrystallized from 2-propanol. The isolated product was dried in vacuum oven at 40° C. to yield 23 g of 30kmSC-PEG.

Preparation of 30kmPEG-Lys(Boc)-OH (Compound 3):

369 mg of H-lys(boc)-OH (3 eq.), 646.5 mg of DIEA (10 eq.) and 15 g of 30kmSCPEG (1 eq.) were mixed in 100 mL DMF and 150 ml DCM. The mixture was stirred at room temperature overnight. The insoluble materials were filtered off. The solvent was removed and the residue was recrystallized from 2-propanol. The isolated product was dried at 40° C. under vacuum to yield 12.8 g of 30kmPEG-Lys(Boc)-OH.

Preparation of 30kmPEG-Lys(Boc)-DBCO (Compound 4):

6 g of 30kmPEG-Lys(Boc)-OH (1 eq.) was dissolved in 60 mL of DCM and cooled to 0-5° C. 221.1 mg of NH2-DBCO (4 eq.) was added followed by 219.6 mg of DMAP (9 eq.) and 230.4 mg of EDC (6 eq). The mixture was stirred at 0-5° C. for 1 hour. The cooling was removed and the reaction was left at room temperature room temperature overnight. Solvent was removed and the residue was recrystallized from 2-propanol. The isolated product was dried under vacuum at 40° C. to yield 5.7 g of 30kmPEG-Lys(Boc)-alkyne.

Preparation of 30kmPEG-Lys-DBCO (Compound 5):

5.7 g of 30kmPEG-lys(Boc)-DBCO was treated with 86 mL of TFA/DCM (1:2) at room temperature for 1 hr. Solvent was removed under vacuum. The residue was recrystallized from ethyl ether/DCM. The isolated product was dried under vacuum at 40° C. to yield 5.5 g of 30kmPEG-Lys-alkyne.

Preparation of 30kmPEG-Lys(Maleimide)-DBCO (Compound 6):

5.5 g of 30kmPEG-lys-DBCO (1 eq.) was dissolved in 55 mL of DCM. 473 mg of DIEA (20 eq) was added followed by 195 mg of NHS-PEG2-Mal (2.5 eq) at 0/5° C. The mixture was stirred at room temperature overnight. Solvent was removed and the residue was recrystallized from 2-propanol. The isolated product was dried under vacuum to yield 5.1 g of 30kmPEG-Lys(maleimide)-DBCO, which can be used for site-specific conjugation to two different proteins.

### Example 2. Preparation of SCACD3IL2 and SCAPDL1IL10

Two cytokine capped single chain antibody fragment proteins in this invention are made accordingly as highlighted in Formula Ib. The first protein of -A₁-L³-C₁ (A₁-L³-C₁) is made of IL2V+uPA substrate+MMP14 substrate+anti-CD3 (SCACD3IL2) and the second protein -A₂-L⁴-C₂ (A₂-L⁴-C₂) is IL10+uPA+MMP14 substrate+anti-PDL1(SCAPDL1IL10). Both proteins are made via recombinant DNA technology in Chinese hamster ovary (CHO) cells with GS knock out using pD2531nt-HDP expression vector containing GS gene (both the cell line and the vector are licensed from Horizon Discovery, Inc). DNAs encoding the first protein (SCACD3IL2) and the second (SCAPDL1IL10) are synthesized and cloned into pD2531nt-HDP expression vector and transfected to CHO-GS(−/−) cells. Stable cell lines with high production capacity were obtained by culturing the cells in medium containing GS inhibitor MSX without the supplement of glutamine. The two scFvs produced by such cell lines were purified by Ni-chelating resin. Pure SCACD3 and SCAPDL1 are obtained via chromatographic process. The amino acid sequences of SCACD3IL2 and SCAPDL1IL10 are listed below.

Amino acid Sequence of SCACD3IL2 (SEQ ID NO: 1):
APASSSTKKTQLQLEHLLLDLQMILNGINNYKNPKLTRMLTAKFAMPKKA
TELKHLQCLEEELKPLEEVLNGAQSKNFHLRPRDLISNINVIVLELKGSE
TTFMCEYADETATIVEFLNRWITFAQSIISTLTGGGSSGGSGDGIPESLR
AGDGIPESLRAGRGIPESLRAGGKGGGSSGGSGGSGRSANAKAGGGSSGG
SDIKLQQSGAELARPGASVKMSCKTSGYTFTRYTMHWVKQRPGQGLEWIG
YINPSRGYTNYNQKFKDKATLTTDKSSSTAYMQLSSLTSEDSAVYYCARY
YDDHYCLDYWGQGTTLTVSSVEGCGSGGSGGSGGSGGVDDIQLTQSPAIM
SASPGEKVTMTCRASSSVSYMNWYQQKSGTSPKRWIYDTSKVASGVPYRF
SGSGSGTSYSLTISSMEAEDAATYYCQQWSSNPLTFGAGTKLELK
Amino acid Sequence of SCAPDL1 IL10
(SEQ ID NO: 2):
SPGQGTQSENSCTHFPGNLPNMLRDLRDAFSRVKTFFQMKDQLDNLLLKE
SLLEDFKGYLGCQALSEMIQFYLEEVMPQAENQDPDIKAHVNSLGENLKT
LRLRLRRCHRFLPCENKSKAVEQVKNAFNKLQEKGIYKAMSEFDIFINYI
EAYMTMKIRNGGGSSGGSGDGIPESLRAGDGIPESLRAGRGIPESLRAGG
KGGGSSGGSGGSGRSANAKAGGGSSGGSDIQMTQSPSSLSASVGDRVTIT
CRASQDVSTAVAWYQQKPGKAPKLLIYSASFLYSGVPSRFSGSGSGTDFT
LTISSLQPEDFATYYCQQYLYHPATFGQGTKVEIKGCGGGSGGGGSGGGG
SEVQLVESGGGLVQPGGSLRLSCAASGFTFSDSWIHWVRQAPGKGLEWVA
WISPYGGSTYYADSVKGRFTISADTSKNTAYLQMNSLRAEDTAVYYCARR
HWPGGFDYWGQGTLVT

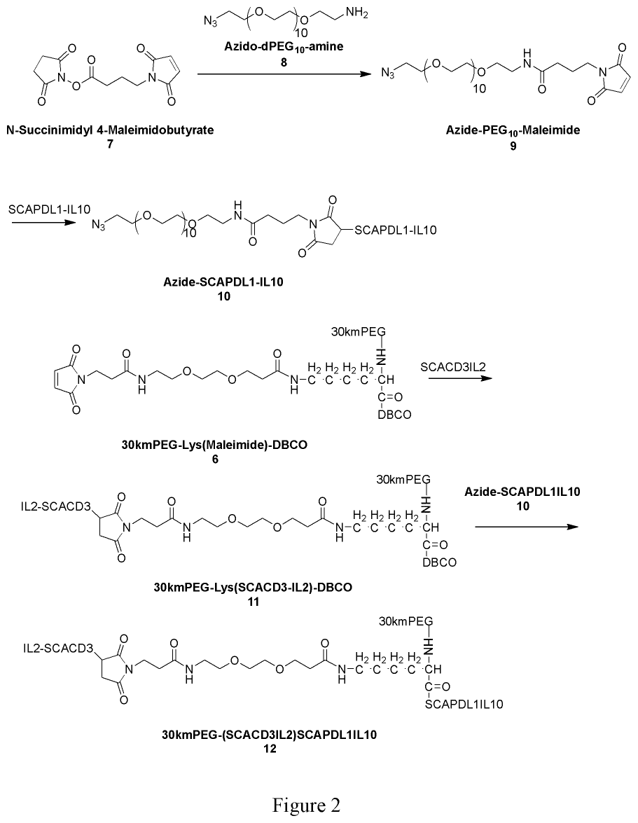

### Example 3. Preparation of 30kmPEG-(SCAPDL1IL10)SCACD3IL2 (FIG. 2)

Preparation of Compound 9:

N-Succinimidyl 4-Maleimidobutyrate (1 eq.) is reacted with Azido-dPEG10-amine (1.5 eq.) in DMSO at room temperature for 45 min. Resulting compound 9 azide-PEG10-Maleimide is used immediately at next step without further purification.

Preparation of Compound 10:

TCEP-HCl (Product #580560, Sigma-Aldrich) is added to SCAPDL1IL10 (5-10 mg/mL) at final 2-10 mM in 200 mM phosphate buffer (pH6.8). The reaction is mixed thoroughly and left at room temperature for 30 min. The reduced SCAPDL1IL10 (1 eq.) is reacted with compound 9 (100 eq.) at room temperature for 1 hr. The reaction is quenched with 10 mM of cystine at room temperature for 10 min. Excess compound 9 is removed by a desalting column in PBS buffer. The fractions of desired compound 10 Azide-SCAPDL1IL10 are pooled and concentrated to 5-10 mg/ml for next step of conjugation.

Preparation of Compound 11:

TCEP-HCl is added to SCACD3IL2 at a final concentration of 2-10 mM in 200 mM phosphate buffer (pH6.8). The reaction is mixed thoroughly and left at room temperature for 30 min. The reduced SCADCD3IL2(1 eq) is reacted with compound 6 (10 eq.) at room temperature for about 2 hours to give compound 11. The crude compound 11 is purified by column. Fractions are collected, pooled and concentrated to 5-10 mg/ml.

Preparation of Compound 12:

Conjugation of compound 11 (1 eq.) with compound 10 (1.33 eq.) is achieved by a clicking chemistry in PBS buffer at room for at least 2 hours.

Purification of target PEGylated bispecific antibody, compound 12, 30kmPEG-(SCAPDL1IL10) SCACD3IL2 is performed in a gel filtration and ion exchange columns. Desired fractions are collected, pooled and concentrated. The target compound is confirmed by SEC-HPLC and cell based activity assay.

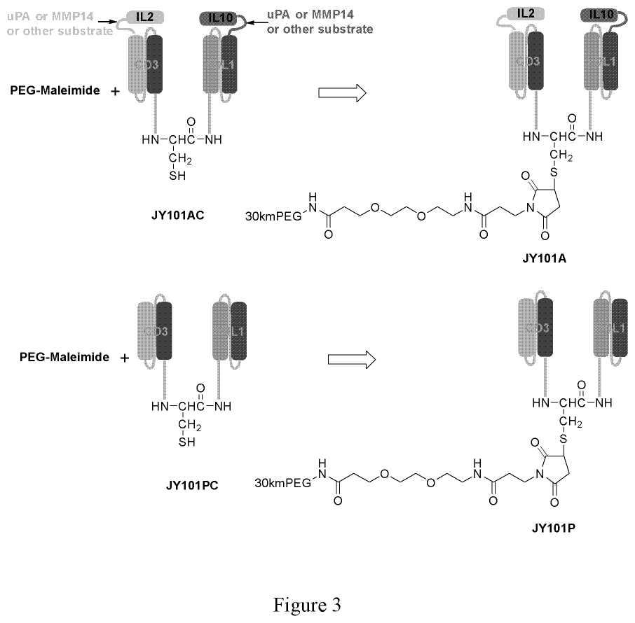

### Example 4. Preparation of JY101A (FIG. 3)

Preparation of Fusion Protein of JY101AC

A fusion protein of two single chain antibody fragment proteins capped with two cytokine (IL2v-SCACD3-SCAPDL1-IL10), JY101AC, was prepared. The structure falls within the scope of Formula II, when C₁=IL2v, L³=uPA substrate or other protease substrate such as MMP14 or a combination of these protease substrates, A₁=SCACD3, L¹ and/or L²=peptide each linked to T (Cysteine) on one peptide terminal and to A₁ or A₂ on the other peptide terminal, A₂=SCAPDL1, L⁴=uPA substrate or other protease substrate such as MMP14 or a combination of these protease substrates, C₂=IL10. This fusion protein was made via recombinant DNA technology in Chinese hamster ovary (CHO) cells with GS knock out using pD2531nt-HDP expression vector containing GS gene (both the cell line and the vector are licensed from Horizon Discovery, Inc). DNA encoding this fusion protein was synthesized and cloned into pD2531nt-HDP expression vector and transfected to CHO-GS(−/−) cells. Stable cell lines with high expression were obtained by culturing the cells in medium containing GS inhibitor Methionine Sulfoximine (MSX) without the supplement of glutamine. The infusion protein produced by such cell lines is purified by Ni-chelating resin, followed by a polishing chromatographic process. The amino acid sequence of JY101AC is listed below.

Amino acid Sequence of JY101AC using MMP14
(SEQ ID NO: 3):
APASSSTKKTQLQLEHLLLDLQMILNGINNYKNPKLTRMLTAKFAMPK
KATELKHLQCLEEELKPLEEVLNGAQSKNFHLRPRDLISNINVIVLEL
KGSETTFMCEYADETATIVEFLNRWITFAQSIISTLTGDGIPESLRAD
IKLQQSGAELARPGASVKMSCKTSGYTFTRYTMHWVKQRPGQGLEWIG
YINPSRGYTNYNQKFKDKATLTTDKSSSTAYMQLSSLTSEDSAVYYCA
RYYDDHYCLDYWGQGTTLTVSSVEGGSGGSGGSGGSGGVDDIQLTQSP
AIMSASPGEKVTMTCRASSSVSYMNWYQQKSGTSPKRWIYDTSKVASG
VPYRFSGSGSGTSYSLTISSMEAEDAATYYCQQWSSNPLTFGAGTKLE
LKGCGGSSGGSDIQMTQSPSSLSASVGDRVTITCRASQDVSTAVAWYQ
QKPGKAPKLLIYSASFLYSGVPSRFSGSGSGTDFTLTISSLQPEDFAT
YYCQQYLYHPATFGQGTKVEIKGGGGSGGGGSGGGGSEVQLVESGGGL
VQPGGSLRLSCAASGFTFSDSWIHWVRQAPGKGLEWVAWISPYGGSTY
YADSVKGRFTISADTSKNTAYLQMNSLRAEDTAVYYCARRHWPGGFDY
WGQGTLVTGDGIPESLRASPGQGTQSENSCTHFPGNLPNMLRDLRDAF
SRVKTFFQMKDQLDNLLLKESLLEDFKGYLGCQALSEMIQFYLEEVMP
QAENQDPDIKAHVNSLGENLKTLRLRLRRCHRFLPCENKSKAVEQVKN
AFNKLQEKGIYKAMSEFDIFINYIEAYMTMKIRNHHHHHH
Amino acid Sequence of JY101AC using uPA
(SEQ ID NO: 4):
APASSSTKKTQLQLEHLLLDLQMILNGINNYKNPKLTRMLTAKFAMPK
KATELKHLQCLEEELKPLEEVLNGAQSKNFHLRPRDLISNINVIVLEL
KGSETTFMCEYADETATIVEFLNRWITFAQSIISTLTGGSGRSANAKA
DIKLQQSGAELARPGASVKMSCKTSGYTFTRYTMHWVKQRPGQGLEWI
GYINPSRGYTNYNQKFKDKATLTTDKSSSTAYMQLSSLTSEDSAVYYC
ARYYDDHYCLDYWGQGTTLTVSSVEGGSGGSGGSGGSGGVDDIQLTQS
PAIMSASPGEKVTMTCRASSSVSYMNWYQQKSGTSPKRWIYDTSKVAS
GVPYRFSGSGSGTSYSLTISSMEAEDAATYYCQQWSSNPLTFGAGTKL
ELKGCGGSSGGSDIQMTQSPSSLSASVGDRVTITCRASQDVSTAVAWY
QQKPGKAPKLLIYSASFLYSGVPSRFSGSGSGTDFTLTISSLQPEDFA
TYYCQQYLYHPATFGQGTKVEIKGGGGSGGGGSGGGGSEVQLVESGGG
LVQPGGSLRLSCAASGFTFSDSWIHWVRQAPGKGLEWVAWISPYGGST
YYADSVKGRFTISADTSKNTAYLQMNSLRAEDTAVYYCARRHWPGGFD
YWGQGTLVTGGSGRSANAKASPGQGTQSENSCTHFPGNLPNMLRDLRD
AFSRVKTFFQMKDQLDNLLLKESLLEDFKGYLGCQALSEMIQFYLEEV
MPQAENQDPDIKAHVNSLGENLKTLRLRLRRCHRFLPCENKSKAVEQV
KNAFNKLQEKGIYKAMSEFDIFINYIEAYMTMKIRNHHHHHH

Preparation of JY101A (PEGylated JY101AC)

Reducing agent (TCEP-HCl, 2-10 mM) is added to IL2v-SCACD3-SCAPDL1-IL10 (JY101AC, 2-10 mg/mL) in 200 mM phosphate buffer at pH6.8. The reaction is mixed thoroughly and left at room temperature for 30 min while stirring. 30kmPEG-maleimide (10 eq.), which is made from the reaction of 30kmPEG-NH2 with NHS-PEG2-maleimide, is added and the mixture is kept at room temperature for 3 hr with gentle stirring. The reaction is quenched by 10 mM cystine at room temperature for 10 min.

Purification of target PEGylated JY101AC (bispecific antibody PEG-IL2v-SCACD3-SCAPDL1-IL10) is performed with cation exchange column (Poros XS) first to remove extra PEG, followed by polish purification through an anion exchange column (Capto Q). Desired fractions are collected, pooled and concentrated. The target compound is confirmed by SEC-HPLC and cell based activity assay.

### Example 5 Preparation of JY101P (PEGylated JY101PC)

Preparation of JY101PC

Similar to JY101AC preparation, JY101PC was prepared using the following amino acid sequence without cytokines:

Amino acid Sequence of JY101P (SEQ ID NO: 5):
DIKLQQSGAELARPGASVKMSCKTSGYTFTRYTMHWVKQRPGQGLEWI
GYINPSRGYTNYNQKFKDKATLTTDKSSSTAYMQLSSLTSEDSAVYYC
ARYYDDHYCLDYWGQGTTLTVSSVEGGSGGSGGSGGSGGVDDIQLTQS
PAIMSASPGEKVTMTCRASSSVSYMNWYQQKSGTSPKRWIYDTSKVAS
GVPYRFSGSGSGTSYSLTISSMEAEDAATYYCQQWSSNPLTFGAGTKL
ELKGCGGSSGGSDIQMTQSPSSLSASVGDRVTITCRASQDVSTAVAWY
QQKPGKAPKLLIYSASFLYSGVPSRFSGSGSGTDFTLTISSLQPEDFA
TYYCQQYLYHPATFGQGTKVEIKGGGGSGGGGSGGGGSEVQLVESGGG
LVQPGGSLRLSCAASGFTFSDSWIHWVRQAPGKGLEWVAWISPYGGST
YYADSVKGRFTISADTSKNTAYLQMNSLRAEDTAVYYCARRHWPGGFD
YWGQGTLVTHHHHHH

JY101P is PEGylated JY101PC. Its preparation is similar to the preparation of JY101A as describe above.

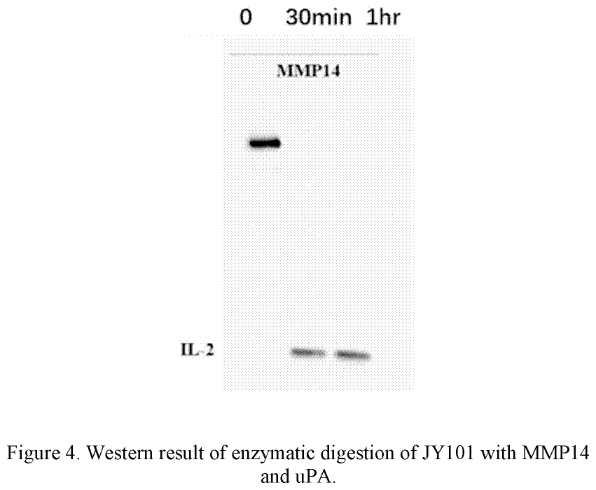

### Example 6. Confirmation of the Cytokines as Parts of the JY101 Fusion Protein Molecules (FIG. 4)

To confirm that the cytokines IL2v and IL10 are components on the above purified fusion protein in Examples 3 and 4, we performed uPA or MMP14 (the linker sequences between the cytokine IL2v and the scFv SCACD3 contains the substrate sites for both enzymes, so does it between IL10 and scFv SCAPDL1) enzyme digestion and detected the digested products by immunoblots probed with anti-IL2 and IL10 antibodies, respectively.

In the digestion reaction, 0.66 ug of JY101-AC was incubated with 33 ng of MMP14 at 37° C. for 30 min or 1 hours in the assay buffer (150 mM NaCl, 50 mM Tris-HCl, 5 mM CaCl2, 0.025% Brij® 35 detergent, pH 7.5) provided by the enzyme manufacturer (Cat. 475936, Merck, Inc). At the end of each reaction time point, SDS-PAGE loading buffer was added to stop the reaction and the samples were boiled at 95° C. for 5 min. After centrifuge at 12000 rpm for 3 min, samples were loaded for SDS-PAGE, and immunoblotting was performed following previously described standard procedures. ECL (enhanced chemiluminescence) reagents were used for signal detection following instruction of the manufacturer.

The results below (FIG. 4) shows that 0.66 ug of JY101-AC fusion protein could be completely digested within 30 minutes by 33 ng of MMP14. Same results were also achieved for 0.66 ug of JY101-AC with the digestion of 33 ng of uPA (Cat. 10815-H08H-A, Sino Biological, Inc) in the assay buffer (50 mM Tris, 0.01% (v/v) Tween®20, pH 8.5) (data not shown here). Besides, these enzyme digestion results also demonstrated that IL2v and IL10 are indeed parts of the expressed fusion protein molecule.

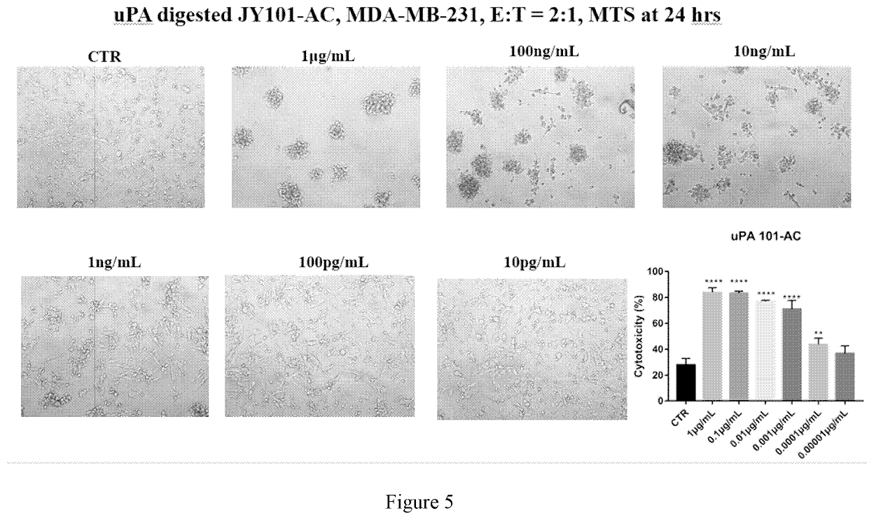

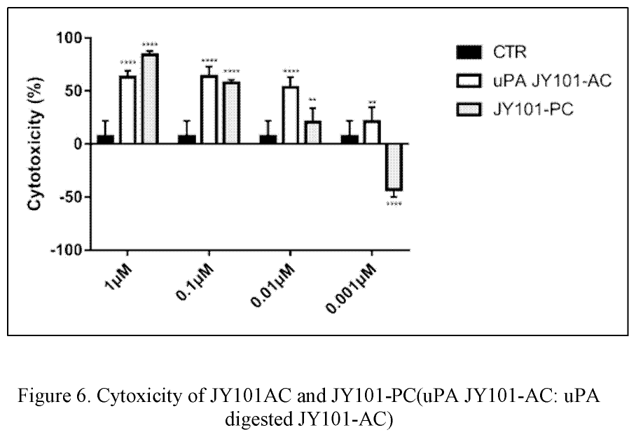

### Example 7. In Vitro Assay Demonstrated Improved Cytotoxicity of Immunocytokine Fused BiTE (FIG. 5 & FIG. 6)

In this assay, peripheral blood lymphocytes (PBMC) from healthy human donors were cultured and proliferated for 1-3 weeks following a T cell expansion protocol provided by the kit manufacturer with some minor modifications (>60% are CD3+ T cells after cell expansion). The T cell expanded PBMC were used as effector cells for the in vitro cytotoxicity assays. 4×10⁴ MDA-MB-231 cells (PDL1 expressing or positive) were seeded in a flat-bottom 96-well plate overnight allowing cells to adhere. On the second day, effector cells were washed, counted, and incubated with indicated doses of JY101AC (uPA digested as described in Example 7) and JY101PC for half an hour at room temperature. Subsequently, effector cells together with the drugs were added at 2:1 effector-to-target (E: T) ratios and incubated at 37° C. for 24 hours. 20 ul MTS (from Promega, Inc) was added into each well according to manufacturer's protocol. Absorbance at OD_{450 nm} was detected and the percentage of dead cells was calculated.

The result as shown in FIG. 5 demonstrated that uPA digested JY101AC is very potent in lysing PD-L1 expressing MDA-MB-231 cells in the presence of effector T cells. At the concentration of 1 ng/ml (E:T ratio of 2:1), the cytotoxicity of uPA digested JY101-AC reached as high as 75%.

For activity comparison, the same molar concentrations of JY101AC (uPA digested) and JY101PC were used in parallel. The results demonstrated significantly higher cytotoxicity for JY101AC (uPA digested) than JY101PC at the low doses (FIG. 6). Since all other conditions are the same, and molar concentrations are the same at each dose, the additional cytotoxicity of JY101AC (uPA digested) above JY101PC should be induced by the uPA released immunocytokines IL2v and IL10.

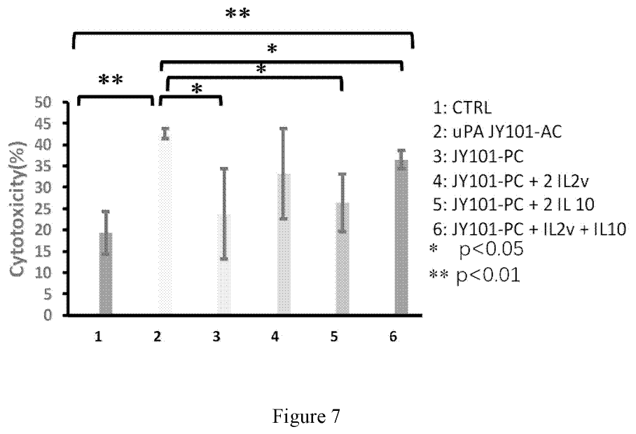

### Example 8. Synergy of Cytotoxicity (FIG. 7)

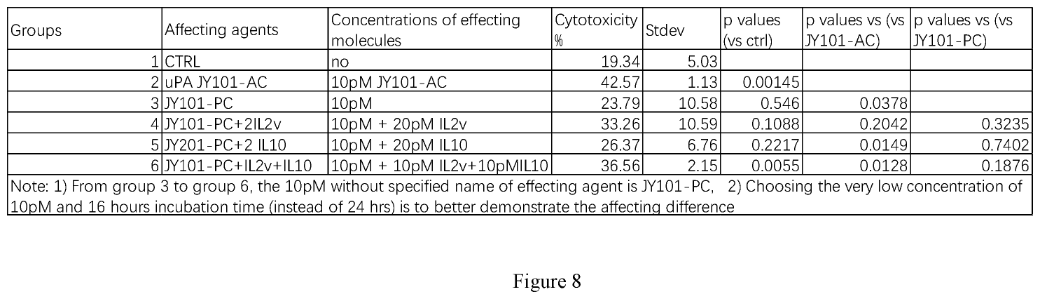

Cytotoxic synergy of JY101AC in vitro is demonstrated in the FIG. 7 and FIG. 8. Similar to experiment procedure in Example 7, expanded T cells in PBMC were activated with 10 pM of JY101AC digested by uPA, JY101PC, IL2V (expressed in house), IL10 (Cat #10947-H07H, Sino Biological, Beijing), or their combinations at the same molar ratio as in JY101AC. A shorter incubation time of 16 hours instead of 24 hours for the drug treatment assured the differences to be easily observed. uPA digested JY101AC not only exerted significantly higher cytotoxicity than JY101PC did to target cells MDA-MB-231, but also induced significantly higher cytotoxicity than JY101PC combined with either 20 pM IL2v or 20 pM IL10. More interestingly, the cytotoxicity induced by uPA digested JY101-AC is significantly higher than 10 pM JY101PC combined with 10 pM IL2v and 10 pM IL10 together. Therefore, the immunocytokines fused BiTE has synergistic effect rather than additive effect in inducing cytotoxicity to target cells. This result provides an extra supportive rational of fusing the selected immunocytokines to BiTE (CD3XPD-L1) and suggests more potency in future therapy, although the mechanism of action awaits for unveiling.

The foregoing examples and description of the preferred embodiments should be taken as illustrating, rather than as limiting the present invention as defined by the claims. As will be readily appreciated, numerous variations and combinations of the features set forth above can be utilized without departing from the present invention as set forth in the claims. Such variations are not regarded as a departure from the scope of the invention, and all such variations are intended to be included within the scope of the following claims. All references cited herein are incorporated by reference herein in their entireties.

1. A compound of Formula (Ib) 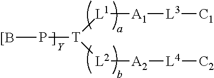
wherein
P is a non-immunogenic polymer;
B is H, a capping group selected from C₁₋₁₀ alkyl and aryl, wherein one or more carbons of said alkyl is optionally replaced with a heteroatom;
A₁ and A₂ are two different antibodies, antibody fragments or single chain antibodies or other forms of antibodies or any combination thereof,
C₁ and C₂ are each a capping group or null;
L³ and L⁴ are each an enzyme cleavable substrate or null;
L¹ and L² are each independently a bifunctional linker;
a and b are independently an integer selected from 0-10;
Y is an integer selected from 0-10.
and
T is a linker moiety.

2. The compound of claim 1, wherein the linkages of T for (L¹)ₐ-A₁ and (L²)_{b}-A₂ are derived from two functional groups independently selected from the group consisting of amine, carboxylic acid, alcohol, thiol, maleimide, azide, alkyne, Dibenzocyclooctyl (DBCO), trans-cyclooctenes, tetrazines, carbonyl, hydrazide, oxime, triarylphosphine, potassium acyltrifluoroborates, and O-carbamoylhydroxylamines.

3. The compound of claim 1, wherein L¹ and L² each comprises a spacer independently selected from the group consisting of —(CH₂)ₘXY(CH₂)ₙ—, —X(CH₂)mO(CH₂CH₂O)ₚ(CH₂)ₙY—, —(CH₂)ₘX—Y(CH₂)ₙ—, —(CH₂)ₘheterocyclyl-, —(CH₂)ₘX—, and —X(CH₂)ₘY—, amino acid and any peptide wherein m, n, and p in each instance are independently an integer ranging from 0 to 25; X and Y in each instance are independently selected from the group consisting of C(═O), CR₁R₂, NR₃, S, O, or Null; wherein R₁ and R₂ independently represent hydrogen, C₁₋₁₀ alkyl or (CH₂)₁₋₁₀(═O), R₃ is H or a C₁₋₁₀ alkyl, and wherein the heterocyclyl is derived from an maleimido or a haloacetyl or a triazolyl or a tetrazolyl group moiety.

4. The compound of claim 1, wherein A₁ is an antibody that binds to a receptor of cytotoxic cell.

5. The compound of claim 1, wherein A₂ is an antibody that binds to an antigen on cancer cells.

6. The compound of claim 1, wherein the antibody is a single chain antibody.

7. The compound of claim 1, wherein A₁ is an anti-CD3 antibody.

8. The compound of claim 1, wherein A₂ is an anti-PDL1 antibody.

9. The compound of claim 1, wherein A₁ is selected from the group consisting of single chain antibody (SCA) fragments of SCACD3 (anti-CD3,), SCACTLA4 (anti-CTLA4), SCAPD1(anti-PD1), LAG3 (anti-LAG3), SCACD40L (anti-CD40L), SCAOX40 (anti-OX40), SCAGITR (anti-GITR), SCAICOS (anti-ICOS), SCACD16(anti-CD16), and SCANKG2D (anti-NKG2D); and A2 is selected from the group of scFvs consisting of anti-HER2, anti-HER3, anti-Nectin-4, anti-CEA, anti-5T4, anti-Cripto-1, anti-EGFR, anti-GRM1, anti-CD47, anti-Siglec-15, anti-Galectin-9, anti-AXL, anti-TRK, anti-FLT3, anti-ALK, anti-ERBB2, anti-CLDN18.2, anti-KIF5B-RET, anti-OX40, anti-Siglec9, anti-Syk, anti-FGFR, anti-KRAS, anti-FGL1, anti-LAG3, anti-Foxp3, anti-CSF-1R, anti-BCMA, anti-SLAMF7, anti-AMHR2, anti-LAIR-1, anti-IL-4R, anti-CD24, anti-SIGLEC10, anti-CD80, anti-VEGF-A, anti-TNFRSF17, anti-TfR1, anti-TNF, anti-STn, anti-SSTR2, anti-RANKL, anti-PSMA, anti-P-cadherin, anti-PcrV, anti-psl, anti-NGF, anti-MSLN, anti-MAPG, anti-Klotho, anti-Interleukin, anti-IGF-1, anti-GPA33, anti-GD2, anti-gp100, anti-Glypican, anti-FAP, anti-EpCAM, anti-EphR, anti-DLL3 and 4, anti-TRAILR2, anti-CD123, anti-CD79B and anti-CD32B, anti-CD64, anti-CD38, anti-Siglec-3, anti-FcγRIIB, anti-TNFRSF8, anti-CD22, 20, 16 and 19, anti-CLEC12A, anti-Cadherins, anti-c-MET, anti-B7-H3, anti-4-1BB, anti-PSMA, anti-B7H3, anti-MUC16, anti-FLT3, anti-Galectin-9, anti-Siglec-15, and anti-GRM1, etc.

10. The compound of claim 1, wherein C₁ and C₂ is independently immunostimulatory cytokine that binds to a receptor of T cells to promote T cell proliferation or null, and a least one of C₁ and C₂ is a capping group.

11. The compound of claim 1, wherein C₁ and C₂ are each independently selected from the group consisting of IL-2, IL-4, IL-10, IL-12, IL-15, and interferon-gamma or null, and a least one of C₁ and C₂ is a capping group.

12. The compound of claim 1, wherein the non-immunogenic polymer comprises a member selected from the group consisting of polyethylene glycol (PEG), dextrans, carbohydrate-base polymers, polyalkylene oxide, polyvinyl alcohols, hydroxypropyl-methacrylamide (HPMA), and a co-polymer thereof.

13. (canceled)

14. The compound of claim 1, wherein the non-immunogenic polymer comprises PEG in a molecular weight ranging from 3000 to 80000.

15. The compound of claim 1, wherein the non-immunogenic polymer comprises a linear or branched polyethylene glycol.

16. The compound of claim 1, wherein the non-immunogenic polymer is polyethylene glycol, and the linkage of the T to P is cleavable.

17. The compound of claim 1, wherein L³ or L⁴ is a substrate of a protease, wherein the protease is selected from the group consisting of a collagenase, a gelatinases, a matrilysin, MMP-12, a membrane type MMP, a stromelysin, a MMP-21, a MMP-27, a disintegrin, a metalloproteina with thrombospondin motif (ADAMTs), a procathepsin B, a cathepsin B, a cathepsin S, a caspase, a chondroitinase, a Hyaluronidase, uPA, and tPA.

18. (canceled)

19. The compound of claim 1, wherein the linkage of T to P is selected from the group consisting of amide, ester, carbamate, carbonate, imide, imine, hydrazones, sulfone, ether, thioether, thioester and disulfide, or wherein the linkage of T to P is derived from a pair of functional groups selected from the group consisting of thiol and maleimide, amine and haloacetyl, carboxylic acid and amine, azide and alkyne, trans-cyclooctene and tetrazine, carbonyl and hydrazide, carbonyl and oxime, azide and triarylphosphine, and potassium acyltrifluoroborates and O-carbamoylhydroxylamines.

20. (canceled)

21. The compound of claim 1, wherein T is derived from a natural or unnatural amino acid selected from the group consisting of Cysteine, Lysine, Asparagine, Aspartic, Glutamic acid, Glutamine, Histidine, Serine, Threonine, Tryptophan, Tyrosine, genetically-encoded alkene lysines, 2-Amino-8-oxononanoic acid, m or p-acetyl-phenylalanine, amino acid bearing a β-diketone side chain, (S)-2-amino-6-(((1R,2R)-2-azidocyclopentyloxy)carbonylamino)hexanoic acid, azidohomoalanine, pyrrolysine analogue N⁶-((prop-2-yn-1-yloxy)carbonyl)-L-lysine, (S)-2-Amino-6-pent-4-ynamidohexanoic acid, (S)-2-Amino-6-((prop-2-ynyloxy)carbonylamino)hexanoic acid, (S)-2-Amino-6-((2-azidoethoxy)carbonylamino)hexanoic acid, p-azidophenylalanine, para-azidophenylalanine, N^{ε}-Acryloyl-1-lysine, Nε-5-norbornene-2-yloxycarbonyl-1-lysine, N-ε-(Cyclooct-2-yn-1-yloxy)carbonyl)-L-lysine, N-ε-(2-(Cyclooct-2-yn-1-yloxy)ethyl) carbonyl-L-lysine, and genetically Encoded Tetrazine Amino Acid.

22. The compound of claim 1, wherein P is derived from a PEG having a terminal maleimide.

23. The compound of claim 1, wherein P is derived from a PEG having a terminal maleimide, T is derived from cysteine, and the linkage between P and T is a thioether.

24. The compound of claim 1, wherein Y=0,
and the compound is

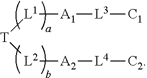

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. The compound of claim 24, wherein
A₁ is SCACD3 and A₂ is SCAPDL1,
C₁ and C₂ are independently IL2v or IL10,
L³ and L⁴ are each independently uPA, MMP14 or null.

38. A pharmaceutical formulation comprising a therapeutically effective amount of the compound of claim 1 and a pharmaceutically acceptable carrier.

39. A method of treating a disease in a subject in need thereof comprising administering an effective amount of the compound of claim 1.

40. The method of claim 39, wherein the disease is cancer selected from the group consisting of breast cancer, ovarian cancer, prostate cancer, lung cancer, pancreatic cancer, kidney cancer, bladder cancer, stomach cancer, colon cancer, colorectal cancer, salivary gland cancer, thyroid cancer and endometrial cancer.

41. The compound of claim 1, and a least one of C₁ and C₂ is a capping group.


SEQUENCE LISTING (5 sequences)

SEQ ID NO: 1 - protein, 445 aa, Artificial Sequence
  SCACD3IL2
APASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLTGGGSSGGSGDGIPESLRAGDGIPESLRAGRGIPESLRAGGKGGGSSGGSGGSGRSANAKAGGGSSGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGCGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 2 - protein, 466 aa, Artificial Sequence
  SCAPDL1 IL10
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRNGGGSSGGSGDGIPESLRAGDGIPESLRAGRGIPESLRAGGKGGGSSGGSGGSGRSANAKAGGGSSGGSDIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKGCGGGSGGGGSGGGGSEVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVT

SEQ ID NO: 3 - protein, 808 aa, Artificial Sequence
  JY101AC using MMP14
APASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLTGDGIPESLRADIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKGCGGSSGGSDIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKGGGGSGGGGSGGGGSEVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTGDGIPESLRASPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRNHHHHHH

SEQ ID NO: 4 - protein, 810 aa, Artificial Sequence
  JY101AC using uPA
APASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLTGGSGRSANAKADIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKGCGGSSGGSDIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKGGGGSGGGGSGGGGSEVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTGGSGRSANAKASPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRNHHHHHH

SEQ ID NO: 5 - protein, 495 aa, Artificial Sequence
  JY101P
DIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKGCGGSSGGSDIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKGGGGSGGGGSGGGGSEVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTHHHHHH